(19) United States
(12) Patent Application Publication
(10) Pub. No.: US 2016/0017313 A1
(43) Pub. Date: Jan. 21, 2016

(54) ANALYSIS OF MRNA HETEROGENEITY AND STABILITY

(71) Applicant: MODERNA THERAPEUTICS, INC., Cambridge, MA (US)

(72) Inventors: Vlad Boris SPIVAK, Medford, MA (US); Zahra SHAHROKH, Weston, MA (US); William Joseph ISSA, Cambridge, MA (US)

(21) Appl. No.: 14/776,893

(22) PCT Filed: Mar. 14, 2014
(86) PCT No.: PCT/US2014/029238

§ 371 Date: Sep. 15, 2015

Related U.S. Application Data
(60) Provisional application No. 61/799,905, filed on Mar. 15, 2013.

(51) Int. Cl. C12N 15/10; B01D 21/26; G01N 27/447

(52) U.S. Cl. CPC C12N 15/101; G01N 27/447; B01D 21/262

(57) ABSTRACT

Reversed phase-High Performance (High Pressure) Liquid Chromatography (RP-HPLC) and Size Exclusion Chromatography (SEC) methods have been developed for monitoring structural and size heterogeneity as well as stability of large RNA transcripts, including lengths of up to at least 10,000 nucleotides. The methods are designed for significantly larger mRNAs that could be monitored in the past, including lengths of up to at least 10,000 nucleotides, and including chemically modified RNA transcripts. SEC techniques are also used in the preparative purification of large RNA transcripts to remove impurities, including hybridized nucleic acid impurities and multimeric RNA species. All of these techniques are also beneficial in that they can be used for large scale manufacturing of therapeutics.

### BACKGROUND OF THE INVENTION

1. Field of the Invention

The present invention relates analysis of mRNA heterogeneity and stability, and specifically to analytical methods for monitoring structural and size heterogeneity, as well as physical-chemical stability, of large mRNAs.

2. Description of the Related Art

RNA transcripts have strong potential as therapeutics, but effective methods of determining the heterogeneity and physical-chemical stability for these RNA transcripts for introduction into the body remains a problem. Methods for determining the heterogeneity and physical-chemical stability of mRNA manufactured as a human therapeutic are needed to demonstrate consistency of the batches and for maintaining safety and efficacy of the therapeutic product during long-term storage. There is little information in the field on analytical methods that are also effective for use in determining heterogeneity and stability indicators for large mRNAs. Furthermore, RNA transcripts of greater than 100 nucleotides are difficult to characterize. For example, tight binding of mRNA to surfaces (e.g., HPLC columns) tends to provide anomalous results, making it difficult to get accurate characterizations of large RNA transcripts.

Purification of mRNAs manufactures as therapeutics can also be a problem. Size exclusion chromatography (SEC) has been previously utilized in the art to purify small scale quantities of in vitro transcribed RNAs of sizes typically less than several hundred nucleotides at lab scale [2] [3]. Short RNAs (less than 400 nucleotides) were able to be separated from plasmid DNA templates, nucleotide triphosphates and other short abort sequences generated during transcription. These results were achieved under non-denaturing conditions. While this has proven to work adequately for separations of short synthetic RNAs or RNA transcripts of less than 400 nucleotides in size, these methods have not been shown to work for longer RNAs or RNA transcripts of greater than 400 nucleotides in length. Purification and characterization techniques for longer RNAs and full length transcripts for use in therapeutics are desirable, preferably techniques that are scalable, reproducible, and thus useable for large scale manufacturing of therapeutics.

### SUMMARY OF THE INVENTION

Reversed phase-High Performance (High Pressure) Liquid Chromatography (RP-HPLC), Size Exclusion Chromatography (SEC), and other methods have been developed for monitoring structural and size heterogeneity as well as stability of large mRNAs. The purpose of these methods is to demonstrate success of the manufacturing process at the molecular level in making the intended product (e.g., a therapeutic including an RNA transcript) by considering the size the product of the manufacturing process and confirming that this size matches the expected size of the target of the manufacturing process. Understanding the heterogeneity (types and percentages of product variants/impurities) of the manufacturing product and the impact of impurities in the product on safety and efficacy, the manufacturing process can be improved.

One challenge, however, is that RNA transcripts of greater than100 nucleotides are difficult to characterize due to, for example, tight binding of mRNA to surfaces (e.g., HPLC columns), which tends to provide anomalous results, making it difficult to get accurate characterizations of large RNA transcripts. The methods of the present invention are designed for significantly larger mRNAs that could be monitored in the past, including lengths of up to at least 10,000 nucleotides. In addition, the methods allow for characterization of chemically modified RNA transcripts, which was also not possible with past techniques.

SEC techniques of the present invention are also used in the preparative purification of RNA transcripts. In addition to removing impurities, such as plasmid DNA templates, nucleotide triphosphates and other short abort sequences generated during transcription, SEC is used in the present invention to remove hybridized nucleic acid impurities and multimeric RNA species. The chromatographic separation may be performed under denaturing conditions (e.g., high temperature), partially denaturing conditions, non-denaturing conditions and may include the use of chaotropic salts. This purification scheme allows for the purification of chemically modified RNA transcripts and RNA transcripts of up to 10,000 nucleotides in length. These SEC techniques are also beneficial in that they can be used for cGMP manufacturing at a large scale, and more specifically at a pilot and process scale, to remove hybridized nucleic acid contaminants from mRNA preparations.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1A is a flow chart illustrating an overview of the SEC methods for analytical chromatography, in accordance with an embodiment of the invention.

FIG. 1B is a flow chart illustrating an overview of the RP-HPLC methods for analytical chromatography, in accordance with an embodiment of the invention.

FIG. 1C is a flow chart illustrating an overview of the SEC methods for purification, in accordance with an embodiment of the invention.

FIG. 1D is a graphical representation of the resolution of GCSFm XuF and ATP on SEC, column run at 75° C., in accordance with an embodiment of the invention.

FIG. 1E is a graphical representation of the resolution of GCSFm XuF and ATP on SEC, column run at 25° C., in accordance with an embodiment of the invention.

FIG. 2A is a graphical representation of the load amount vs. temperature, SEC run at 25° C., in accordance with an embodiment of the invention.

FIG. 2B is a graphical representation of the load amount vs. temperature, SEC run at 65° C., in accordance with an embodiment of the invention.

FIG. 2C is a graphical representation of the load amount vs. temperature, SEC run at 75° C., in accordance with an embodiment of the invention.

FIG. 2D is a graphical representation of the shoulder peak area vs. temperature trend, in accordance with an embodiment of the invention.

FIG. 2E is a graphical representation of the main peak area vs. temperature, in accordance with an embodiment of the invention.

FIG. 3A is a graphical representation of the effect of mobile phase ionic strength, SEC run at 25° C., in accordance with an embodiment of the invention.

FIG. 3B is a graphical representation of the effect of mobile phase ionic strength, SEC run at 75° C., in accordance with an embodiment of the invention.

FIG. 4 is a graph illustrating the temperature dependence of the leading shoulder peak, in accordance with an embodiment of the invention.

FIG. 5A is a graphical representation of the effect of sample heat denaturation on the leading shoulder, SEC run at 75° C., in accordance with an embodiment of the invention.

FIG. 5B is a graphical representation of the effect of sample heat denaturation and re-equilibration to room temperature on the leading shoulder, SEC run at 25° C., in accordance with an embodiment of the invention.

FIG. 6A is a graphical representation of the time course of SEC change at 37° C., water formulation, in accordance with an embodiment of the invention.

FIG. 6B is a graphical representation of the time course of SEC change at 37° C., 5% sucrose formulation, in accordance with an embodiment of the invention.

FIG. 6C is a graphical representation of the time course of SEC change at 37° C., PBS formulation, in accordance with an embodiment of the invention.

FIG. 6D is a graphical representation of the time course of SEC change at 37° C., NaCitrate formulation, in accordance with an embodiment of the invention.

FIG. 7 is a graphical representation of improved purity of mRNA as a function of manufacturing process as determined by SEC, in accordance with an embodiment of the invention.

FIG. 8 is a graphical representation of the effect of nucleotide chemistry on the purity of the mRNA, in accordance with an embodiment of the invention.

FIG. 9A is a graphical representation of RP-HPLC water, day 0 parameters injected onto a WATERS XBRIDGE™ C18 50 mm column at 35° C., in accordance with an embodiment of the invention

FIG. 9B is a graphical representation of RP-HPLC, 37° C. stability, water, day 7 parameters injected onto a WATERS XBRIDGE™ C18 50 mm column at 35° C., in accordance with an embodiment of the invention.

FIG. 9C is a graphical representation of RP-HPLC, sucrose, day 7 parameters injected onto a WATERS XBRIDGE™ C18 50 mm column at 35° C., in accordance with an embodiment of the invention.

FIG. 9D is a graphical representation of RP-HPLC, 37° C. PBS, day 7 parameters injected onto a WATERS XBRIDGE™ C18 50 mm column at 35° C., in accordance with an embodiment of the invention.

FIG. 9E is a graphical representation of RP-HPLC, citrate, day 7 parameters, in accordance with an embodiment of the invention.

FIG. 10A is a graphical representation of RP-HPLC profile of intact GCSF modified mRNA isolated by the P1 purification process injected onto a WATERS XBRIDGE™ C18 50 mm column at 35° C., in accordance with an embodiment of the invention.

FIG. 10B is a graphical representation of RP-HPLC profile of intact GCSF modified mRNA isolated by the P2 purification process injected onto a WATERS XBRIDGE™ C18 50 mm column at 35° C., in accordance with an embodiment of the invention.

FIG. 11A is a graphical representation of RP-HPLC profile of intact GCSF mRNA synthesized with G0 chemistry injected onto a WATERS XBRIDGE™ C18 50 mm column at 35° C., in accordance with an embodiment of the invention.

FIG. 11B is a graphical representation of RP-HPLC profile of intact GCSF modified mRNA synthesized with G1 chemistry injected onto a WATERS XBRIDGE™ C18 50 mm column at 35° C., in accordance with an embodiment of the invention.

FIG. 11C is a graphical representation of RP-HPLC profile of intact GCSF modified mRNA synthesized with G2 chemistry injected onto a WATERS XBRIDGE™ C18 50 mm column at 35° C., in accordance with an embodiment of the invention.

### DETAILED DESCRIPTION OF THE INVENTION

Definitions

Terms used in the claims and specification are defined as set forth below unless otherwise specified.

The term “approximately” or “about” means +/−10% of the recited value.

The terms “associated with,” “conjugated,” “linked,” “attached,” “coupled,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used.

The term “chaotropic agent” is a substance that denatures the secondary and/or tertiary structure of molecule(s) by disrupting the intramolecular and intermolecular hydrogen bonding of biological materials or macromolecules, such as proteins and nucleic acids. A chaotropic salt is an example of such an agent.

The terms “characterize” or “characterizing,” when used with regard to a polynucleotide (e.g., RNA transcript) or impurities separated using methods described herein, refers to determining information about the polynucleotide or one or more of the impurities, such as determining information about or quantifying charge variants, size or structural heterogeneity, physical-chemical stability, structural isoforms, among other aspects associated with the polynucleotide or one or more of the impurities.

The term “denaturing conditions” refers to conditions that cause a biological material or macromolecule, such as a nucleic acid or protein, to lose a structure (e.g., a tertiary structure or secondary structure) that is present in its native state, by application of some external stress or compound, such as a chaotropic agent, a concentrated inorganic salt, or heat (thermal denaturing conditions). “Non-denaturing conditions” are conditions that do not cause the biological material to lose this structure. “Partially denaturing conditions” are conditions that cause the biological material to lose at least a portion of this structure.

The term “DNA template” refers to a polynucleotide template for RNA polymerase. Typically a DNA template includes the sequence for a gene of interest operably linked to a RNA polymerase promoter sequence.

The term “eluent” refers to a carrier portion of the mobile phase, such as a solvent or mixture of solvents with which a sample can be delivered in a chromatographic process.

The term “eluate” refers to the material that emerges from or is eluted from a chromatographic process.

The term “impurities” or “contaminants” refers to unwanted components, material defilement, admixture, byproducts of a reaction, or imperfections in a sample. For example, impurities removed in a purification of a long or full-length RNA transcript can include short transcripts, DNA template utilized during in vitro transcription, hybridized nucleic acid impurities, and process related impurities (e.g., enzymes, endotoxin, nucleotides, small molecules, etc.).

The term “in vitro” refers to events that occur in an artificial environment, e.g., in a test tube or reaction vessel, in cell culture, in a Petri dish, etc., rather than within an organism (e.g., animal, plant, or microbe).

The term “mobile phase” refers to a phase or portion that moves in a chromatographic method, such as by passing through a column, and it includes the sample and the eluent.

The term “modified” or “chemically modified” refers to a changed state or structure of a molecule of the invention. Molecules may be modified in many ways including chemically, structurally, and functionally. In one embodiment, the RNA transcripts of the present invention are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

The term “native” or “naturally occurring” as used throughout the specification in connection with biological materials such as polypeptides, nucleic acids, host cells, and the like, refers to materials which are found in nature or a form of the materials that is found in nature.

The terms “purify,” “purified,” “purification” means to be or make substantially pure or clear from unwanted components, material defilement, admixture or imperfection.

The term “RNA transcript” refers to a ribonucleic acid produced by an in vitro transcription reaction using a DNA template and an RNA polymerase. As described in more detail below, an RNA transcript typically includes the coding sequence for a gene of interest and a poly A tail. RNA transcript includes an mRNA. The RNA transcript can include modifications, e.g., modified nucleotides. As used herein, the term RNA transcript includes and is interchangeable with mRNA, modified mRNA “mmRNA” or modified mRNA, and primary construct.

The term “substantially pure” means that the described species of molecule is the predominant species present, that is, on a molar basis it is more abundant than any other individual species in the same mixture. In certain embodiments, a substantially pure molecule is a composition wherein the object species comprises at least 50% (on a molar basis) of all macromolecular species present. In other embodiments, a substantially pure composition will comprise at least 80%, 85%, 90%, 95%, or 99% of all macromolecular species present in the composition. In other embodiments, the object species is purified to essential homogeneity wherein contaminating species cannot be detected in the composition by conventional detection methods and thus the composition consists of a single detectable macromolecular species.

The term “sample” refers to a subset of the tissues, cells or component parts of an organism, such as nucleic acids, proteins, body fluids, etc. or a homogenate, lysate or extract prepared from a whole organism or a subset of its tissues, cells or component parts, or a fraction or portion thereof. A sample further refers to a medium or phase, such as a nutrient broth or gel or other delivery agent, which may contain cellular components, such as proteins or nucleic acid molecules.

The terms “scalable” or “large scale” when used in terms of processes or methods that are scalable or for large scale use refer to processes or methods that are readily useable or readily adaptable for use in a standard cGMP large scale production or manufacturing facility for generating compounds, such as drugs or therapeutics.

The term “sorbent” refers to a material to which one or more components of the sample (e.g., the RNA transcript and/or impurities) adsorb.

The term “solid phase media” or “stationary phase” refers to the phase or portion that is fixed in place or stationary in a chromatographic process, such as a solid material within a column through which the mobile phase passes.

The term “therapeutic agent” or “therapeutic” refers to any agent that, when administered to a subject, has a therapeutic, diagnostic, and/or prophylactic effect and/or elicits a desired biological and/or pharmacological effect.

Those skilled in the art will recognize, or be able to ascertain using no more than routine experimentation, many equivalents to the specific embodiments in accordance with the invention described herein. The scope of the present invention is not intended to be limited to the above, but rather is as set forth in the appended claims.

It must be noted that, as used in the specification and the appended claims, the singular forms “a,” “an” and “the” include plural referents unless the context clearly dictates otherwise.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those well known and commonly used in the art. Standard techniques can be used for chemical syntheses, chemical analyses, pharmaceutical preparation, formulation, and delivery, and treatment of patients.

Methods of the Invention

It is of great interest in the fields of therapeutics, diagnostics, reagents and for biological assays to be able to deliver a nucleic acid, e.g., a ribonucleic acid (RNA) transcript inside a cell, whether in vitro, in vivo, in situ or ex vivo, such as to cause intracellular translation of the nucleic acid and production of an encoded polypeptide of interest. Examples of modified RNA, e.g., RNA transcripts, e.g., mRNA, are disclosed in the following which is incorporated by reference for all purposes: U.S. patent application Ser. No. 13/791,922, “MODIFIED POLYNUCLEOTIDES FOR THE PRODUCTION OF BIOLOGICS AND PROTEINS ASSOCIATED WITH HUMAN DISEASE,” filed Mar. 9, 2013.

The RNA transcript to be delivered must be separated from the sample such that impurities from the RNA transcript sample are removed and a purified RNA transcript sample is delivered. In addition, it is beneficial to be able to characterize the components of the sample, including the RNA transcript and one or more of the impurities, including determining information about or quantifying charge variants, size or structural heterogeneity, physical-chemical stability, structural isoforms, among other aspects. This information can be used in developing a better therapeutic based on the RNA transcript.

Chromatographic and other characterization or purification methods have been developed in the present invention for purifying and for monitoring structural and size heterogeneity as well as stability of large RNA transcripts or chemically modified RNA transcripts. The purpose of these methods is to demonstrate success of the manufacturing process at the molecular level in making the intended product through confirmation of the mass of product. Success in manufacturing a therapeutic, such as a drug for treatment of a human, at the molecular level can be accomplished by considering the size the product of the manufacturing process and confirming that this size matches the expected size of the target of the manufacturing process. For example, by understanding the heterogeneity (types and percentages of product variants/impurities) of the manufacturing product and the impact of impurities in the product on safety and efficacy, the manufacturing process can be improved. Methods for determining the heterogeneity and physical-chemical stability of RNA transcripts manufactured as a human therapeutic are also needed to demonstrate consistency of the batches and maintaining safety and efficacy of the product during long-term storage. There is little information in the field on analytical methods that are also are indicators for stability for large RNA transcripts.

One challenge, however, is that RNA transcripts of greater than100 nucleotides are difficult to characterize unlike siRNA for which there are many methods available. For example, tight binding of mRNA to surfaces (e.g., HPLC columns) tends to provide anomalous results, making it difficult to get accurate characterizations of large RNA transcripts. Large RNA transcripts potentially have a large overall negative charge, thus potentially facilitating a tight binding to anion exchange resin and no binding to cation exchange resin. The potential very tight binding of RNA to anion exchange resin has been overcome by endeavoring on an extensive anion exchange resin screening and by eluting with high salt, according to some embodiments. The methods of the present invention also address issues with tight binding of the RNA transcript to surfaces through use of chaotropic agents, denaturing conditions, large pore size columns, addition of low level solvents, such as ethanol or acetonitrile to the mobile phase, among other ways.

The methods of the present invention allow for characterization of longer RNA transcripts than has been possible in the past. In some embodiments, the method allows for purification/characterization of RNA transcripts of 300 to 10,000 nucleotides in length, including RNA transcripts in the following ranges: 500 to 10,000 nucleotides, 550 to 10,000 nucleotides, 600 to 10,000 nucleotides, 700 to 10,000 nucleotides, 800 to 10,000 nucleotides, 900 to 10,000 nucleotides, 1,000 to 10,000 nucleotides, 5,000 to 10,000 nucleotides, or any ranges or values within these. In some embodiments, the method allows for purification/characterization of RNA transcripts in preferred ranges of 700 to 3,000 nucleotides, or of 800 to 2,000 nucleotides in length. This is significantly larger than has been possible in the past with other techniques. In some embodiments, the RNA transcript is a full length transcript. As used herein, the term “large RNA transcript” or “long RNA transcript” refers to any RNA transcript falling within any of the ranges described here, including a full length transcript.

The methods of the present invention also allow for characterization or purification of native RNA transcripts or RNA transcripts that are chemically modified. Various modifications are made to RNA transcripts used as therapeutics to, for example, avoid eliciting an immune response to the therapeutic. Examples of chemical modifications that might be made to RNA transcripts are provided in U.S. patent application Ser. No. 13/791,922, “MODIFIED POLYNUCLEOTIDES FOR THE PRODUCTION OF BIOLOGICS AND PROTEINS ASSOCIATED WITH HUMAN DISEASE,” filed Mar. 9, 2013, which is incorporated by reference for all purposes. These chemically modified RNA transcripts can be purified and/or characterized using the methods of the present invention.

Each of the methods provided below include various steps, and one or more of those steps can be performed under denaturing conditions, partially denaturing conditions, or non-denaturing conditions. The denaturing conditions can include conditions that cause denaturing of the RNA transcript due to temperature, chaotropic agents (including salts), organic agents, among other mechanisms for denaturing. With thermal denaturing conditions, an elevated temperature can be applied. The elevated temperature can be one that is sufficient to denature intramolecular hydrogen bonds, to cause a change in or loss of secondary or tertiary structure, and so forth. For example, the temperature or thermal denaturing conditions can include a temperature of 25 degrees Celsius to 95 degrees Celsius, 35 to 85 degrees Celsius, 55 to 75 degrees Celsius, or of another range within those ranges. Similarly, higher or lower temperatures can be used as appropriate to cause the desired level of denaturing. The temperature or thermal denaturing conditions can also be dependent on the identity of the RNA transcript, such that different temperatures are used for different RNA transcripts or types of RNA transcripts. In some embodiments, the sample or the mobile phase (or a component of the mobile phase) is pre-incubated (before loading) at an elevated temperature sufficient to denature intramolecular hydrogen bonds. The denaturing conditions can also include using chaotropic agents, such as lithium perchlorate and other perchlorate salts, guanidinium chloride and other guanidinium salts, urea, butanol, ethanol, lithium acetate, magnesium chloride, phenol, propanol, sodium dodecly sulfate, thiourea, among others. The denaturing conditions can further include organic denaturing agents, such as dimethyl sulfoxide (DMSO), acetonitrile, and glyoxal. In addition, the denaturing conditions can include a combination of two or more of these types of denaturing conditions. Any one or more of the steps described in the methods below can be performed at an elevated temperature or at ambient temperature, with or without chaotropic or organic agents, and so forth.

Each of the methods below includes a stationary phase through which the mobile phase passes. A variety of different materials can be used as the stationary phase in any of the methods, including a variety of different particle and pore sizes. Particle sizes can include standard sizes used in chromatography methods, including sizes in the range of less than 1 μm or 1 to 100 μm (e.g., 5, 10, 20, 50, or 75 μm), or any number or fractional number in between, or any range including or within these numbers. Larger or smaller sizes can also be used. Particles can include small silica beads or other types of particles. Pore sizes can include sizes that are greater than 500 Angstroms, or greater than 600, 700, 800, 900, or 1,000 Angstroms, or any number or fractional number in between, or any range including or within these numbers. Smaller pore sizes can also be used, such as 1 to 500 Angstroms.

SEC for Analysis of Structural Isoforms, Degradation Products, and Size

Size exclusion chromatography (SEC) is a method in which molecules in solution are separated by their size, and in some cases molecular weight. SEC typically uses porous particles as a stationary phase to separate molecules of different sizes in a mobile phase. Molecules that are smaller than the pore size can enter the particles and therefore have a longer path and longer transit time across the stationary phase than larger molecules that cannot enter the particles.

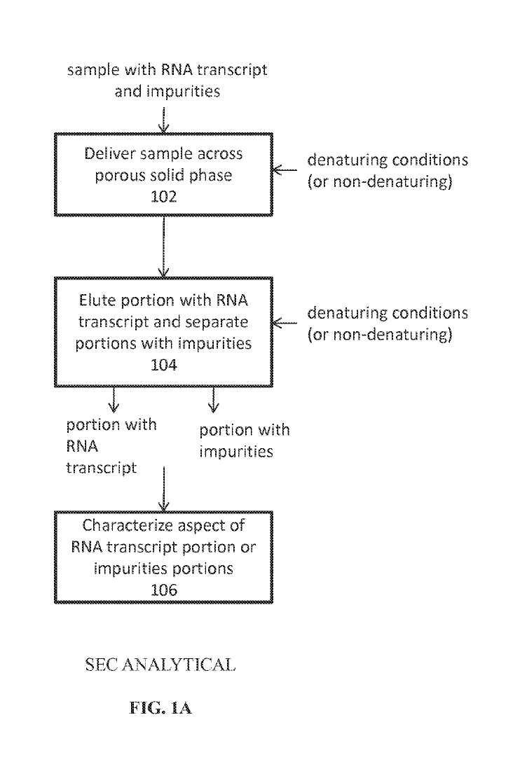

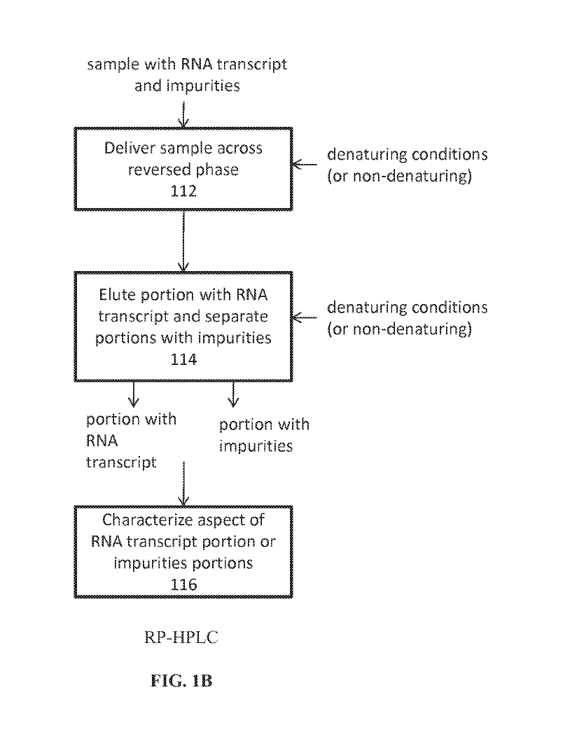

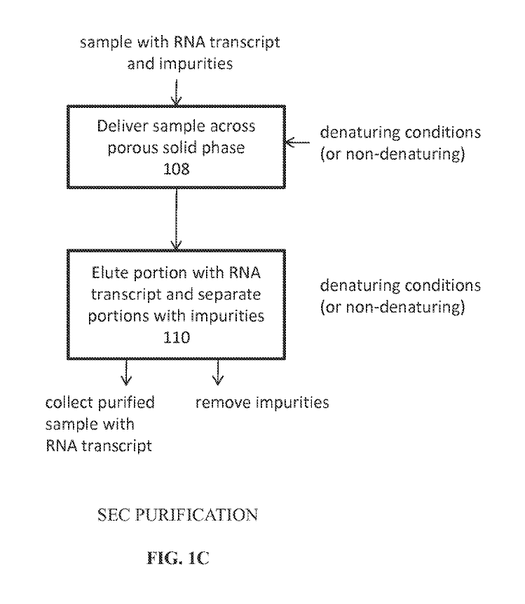

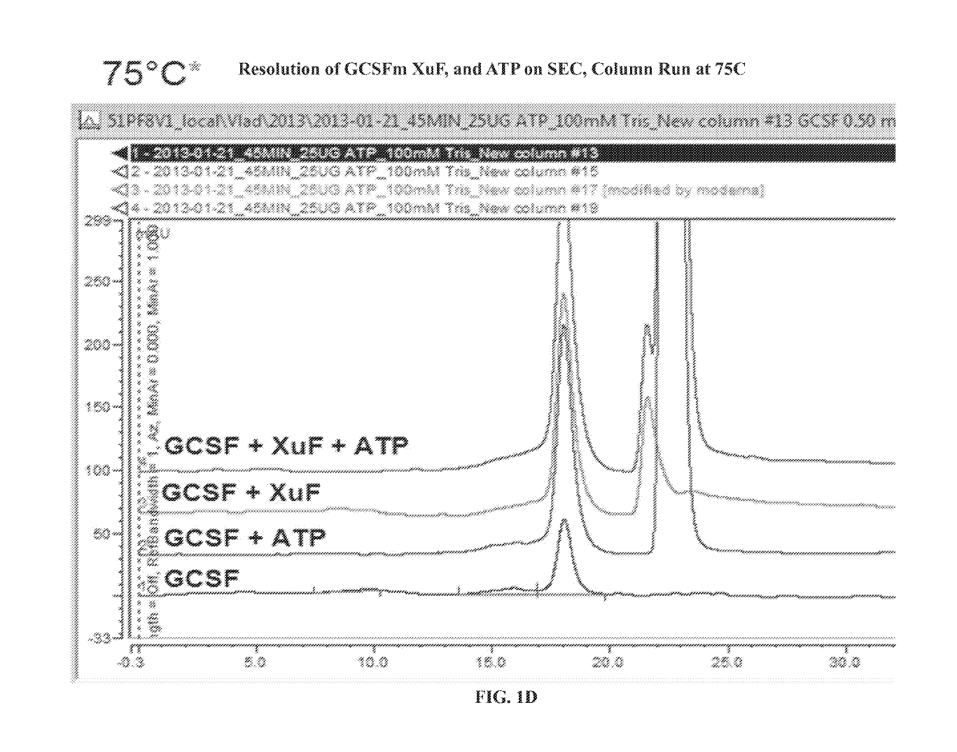

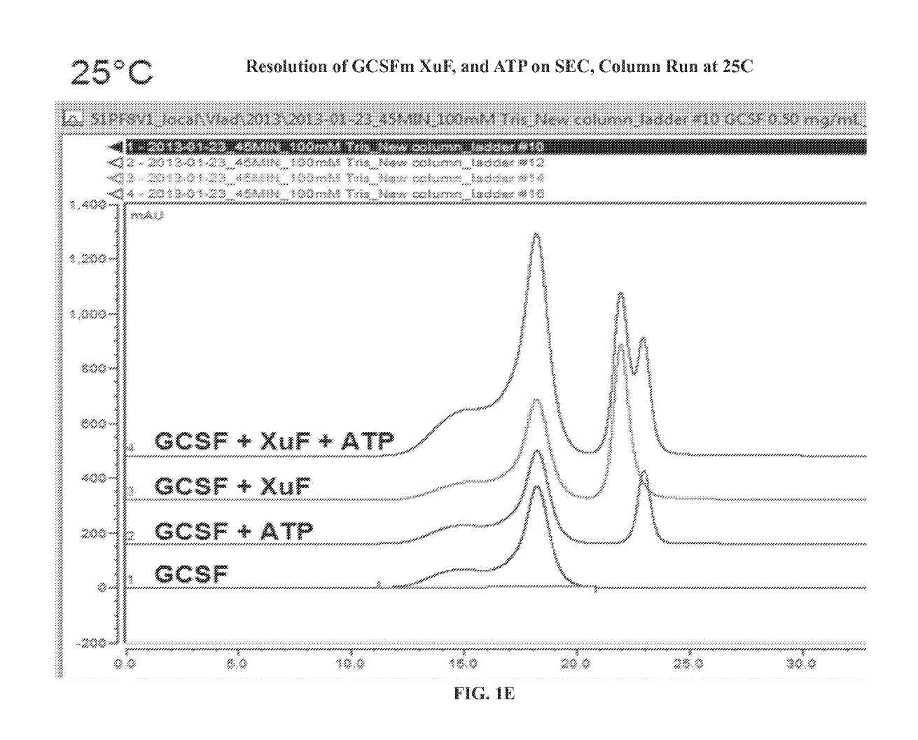

One embodiment of the present invention is a method (e.g., SEC) for characterizing an aspect of a sample comprising an RNA transcript and impurities, as is shown in FIG. 1A. The method comprises delivering 102 (or contacting or loading) the sample across a stationary phase comprising a plurality of pores. The sample is delivered with at least one mobile phase, and the mobile phase can include an eluent selected to effect separation of the components of the sample. The RNA transcript is a different size than the impurities, and the pores are of a size that permits the RNA transcript to elute through the stationary phase at rate that is different from a rate at which the impurities elute through the stationary phase. The method also includes eluting 104 from the stationary phase at least one of the portion of the sample comprising the RNA transcript and one or more separate portions of the sample comprising the impurities. In addition, the method includes characterizing 106 an aspect of the portion of the sample comprising the RNA transcript and the one or more separate portions of the sample comprising the impurities. In one embodiment, SEC was used at 25° C. and 75° C. to discover conformational information about the RNA transcript.

Any of the stationary or mobile phases described above can be used with this method. In one embodiment, a TSKgel G-DNA-PW, Part No. 08032 column is used. In addition, any of these steps can be performed under denaturing conditions, non-denaturing conditions, or partially denaturing conditions, as described above. These methods are described in more detail in the Examples section.

Analytical Reversed Phase-High Performance/Pressure Liquid Chromatography

The present invention also includes methods of RP-HPLC for characterization of samples. One embodiment of the present invention includes a method (e.g., RP-HPLC) for characterizing an aspect of a sample comprising a RNA transcript and impurities, as is shown in FIG. 1B. The method includes delivering 112 the sample across a reversed phase that is a stationary phase. The sample delivered with at least one mobile phase. The RNA transcript and the impurities in the sample interact with the reversed phase to different degrees such that the RNA transcript elutes through the reversed phase at rate that is different from a rate at which each of the impurities elute through the reversed phase. The method also includes eluting 114 from the reversed phase a portion of the sample comprising the RNA transcript and one or more separate portions of the sample comprising the impurities. In addition, the method includes characterizing 116 an aspect of the portion of the sample comprising the RNA transcript or the portions of the sample comprising the impurities. One embodiment includes characterizing by monitoring absorbance at 260 nm and quantifying each peak to get the percentage of each impurity.

Any of the stationary and mobile phases described above can be used in this method, as well. For example, the column used with this method can be a PHENOMENEX® CLARITY OLIGO RP or a WATERS XBRIDGE™ A C18. Mobile phases can include methanol, acetonitrile, and a variety of other components, including non-methanol and non-acetonitrile mobile phases.

Any of the steps described above, including the delivering 112, eluting 114, and the characterizing 116 steps can be performed under denaturing conditions, non-denaturing conditions, or partially denaturing conditions. These methods are described in more detail in the Examples section.

Preparative Size Exclusion Chromatography for Purification

The present invention also includes methods for preparative purification of a sample using size exclusion chromatography (SEC). One embodiment of the method in includes purifying a sample comprising a ribonucleic acid (RNA) transcript and impurities, as shown in FIG. 1C. The method comprises delivering 108 (or contacting or loading) the sample across a stationary phase comprising a plurality of pores. The sample is delivered with at least one mobile phase, and the mobile phase can include an eluent selected to effect separation of the components of the sample. The RNA transcript is a different size than the impurities, and the pores are of a size that permits the RNA transcript to elute through the stationary phase at rate that is different from a rate at which the impurities elute through the stationary phase. The method also includes eluting 110 from the stationary phase a purified sample comprising the RNA transcript.

Any of the stationary and mobile phases described above can be used in this preparative purification method, as well. For example, the stationary phase can be a porous media, including any of poly styrene divinylbenzene, polymethacrylate, crosslinked agarose, allyl dextran with N-N-bis acrylamide, silica, dextran, polyacrylamide, hydrophilic media, and hydrophobic media. The pore and particle sizes described above regarding the SEC methods also apply here, as well.

Any of the steps described above, including the delivering 108 and the eluting 110 steps can be performed under denaturing conditions, non-denaturing conditions, or partially denaturing conditions. These methods are described in more detail in the Examples section.

Other Methodologies to Assess Structural Heterogeneity and Stability

Other methods can also be used to assess structural heterogeneity and stability. These methods include obtaining the RNA transcript, such as a large RNA transcript or one that is chemically modified, and characterizing the RNA transcript using a procedure, such as chip-based capillary electrophoresis, agarose gel electrophoresis, analytical ultracentrifugation, and field flow fractionation. These methods are described in more detail in the Examples section.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

### Example 1

### SEC for Analysis of Structural Isoforms, Degradation Products, and Size

General Summary

An SEC method was developed to monitor the size distribution of RNA transcripts containing modified nucleotides and manufactured by in vitro transcription. The method used a TSKgel G-DNA-PW column that is designed for the separation of large polynucleotides of 500-5000 base pairs.

The mobile phase listed in Table 1 includes EDTA to minimize divalent cation-induced self-association that could lead to peak broadening or particulates that could clog the column. There was little influence of ionic strengths or use of phosphate buffer at neutral pH compared to Tris. Increasing the number of columns placed in tandem increased resolution and run time but did not affect the distribution and relative differences observed between samples.

TABLE 1
SEC-UV Method Summary

| | |
|---|---|
| Column* | 2x TSKgel G-DNA-PW |
| Column Heater | 25° C. (native) & 75° C. (denatured) |
| Mobile Phase | 100 mM Tris, 50 mM NaCl, 1 mM EDTA, pH |
| | 7.4 |
| Gradient | Isocratic |
| Flow Rate | 0.50 mL/min |
| Injection Volume | 50 μL nominal (10-250 μL possible) |
| Nominal Injection | 25 μg |
| Amount | |
| Detection Wavelength | 260 nm |
| Total Run Time | 60 min |

*Note - Some data obtained during SEC assay development with use of one column, rather than two; and a total run time of 45 minutes, rather than 60 minute. These changes to number of column used in series and total run time should have no qualitative effect on the samples analyzed nor on the data obtained.

The method's resolution was evaluated between GCSF mRNA (˜900 nt), XuF primer (˜25 nt), and ATP monomer. FIG. 1D shows that the large mRNA is separated from a 25 nt mRNA, but the 25 nt and ATP are not baseline resolved, even though the column was run at 75° C., where dissociation of oligonucleotide hydrogen bonds occurs. Interestingly, when the column was run at 25° C., as shown in FIG. 1E, an earlier eluting broad shoulder was observed for GCSF mRNA. Since retention time on SEC is dependent on both size and conformation, this earlier eluting shoulder is either higher molecular weight aggregates or a different conformation that gives an apparently larger hydrodynamic radius (e.g., more asymmetrical, side protrusions).

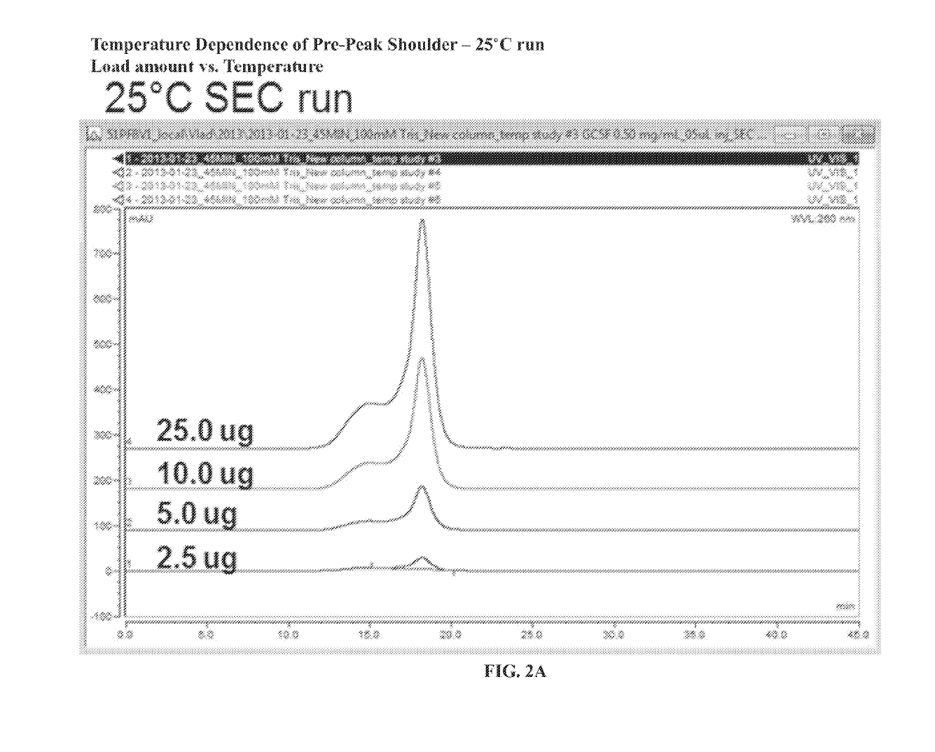

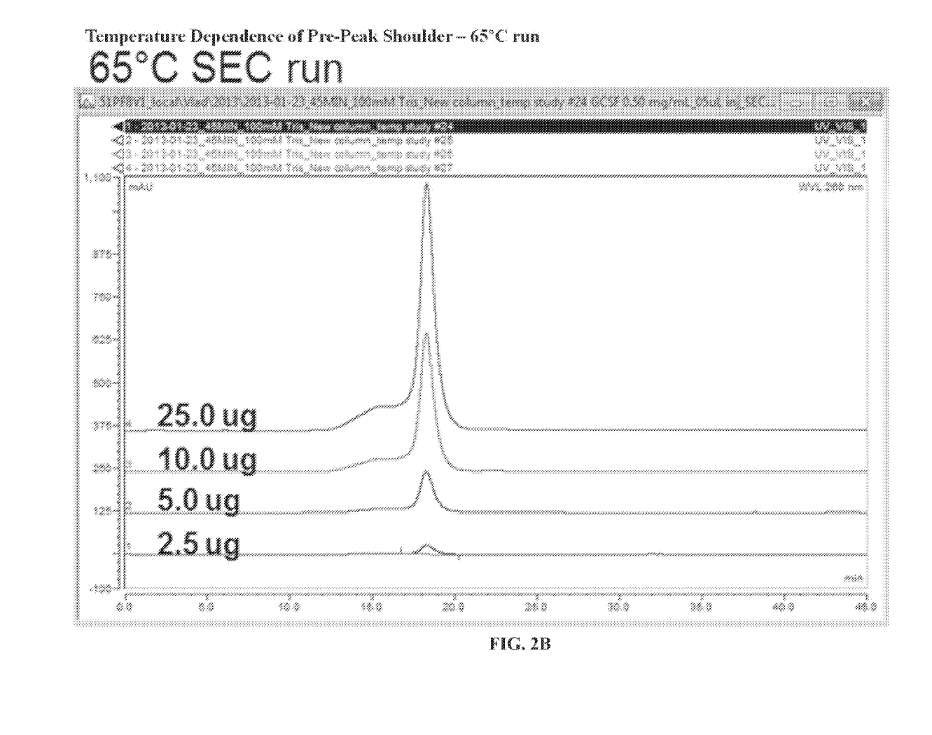

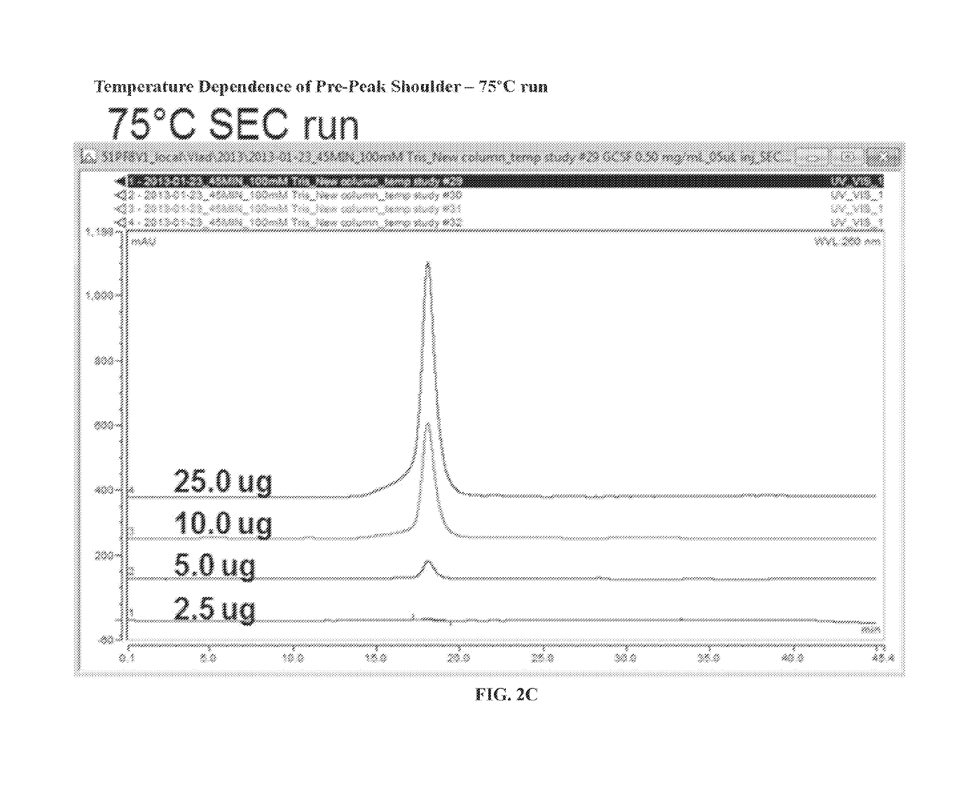

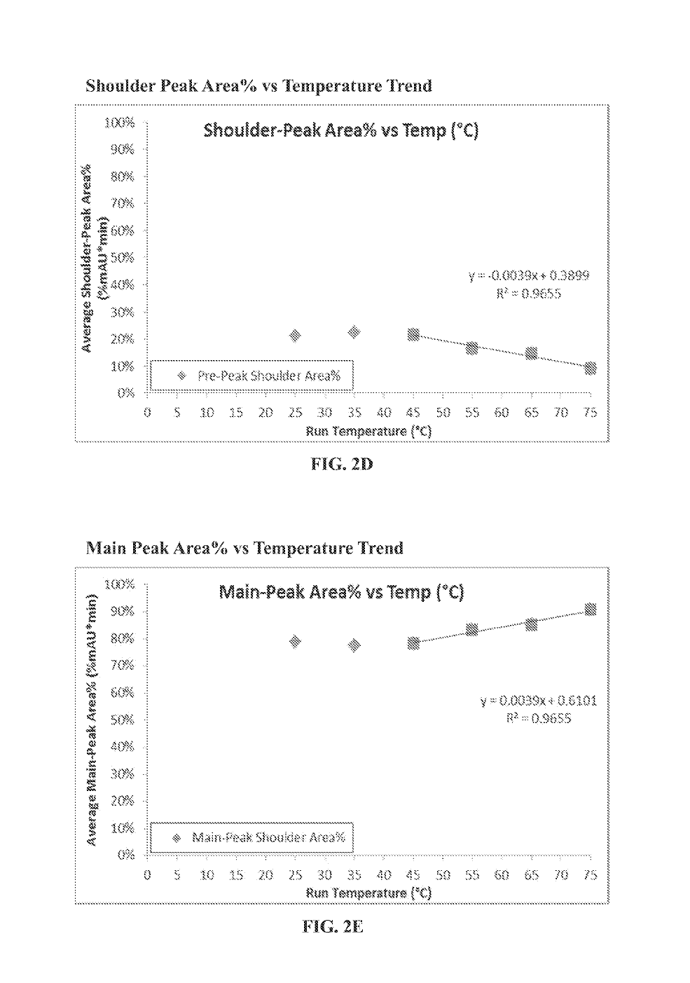

Evaluation of the impact of mRNA load (FIG. 2), given the temperature-dependent presence of a shoulder peak, a study was conducted to evaluate the shoulder peak as a function of temperature. The leading shoulder peak area was found to progressively decrease from ˜25% of total peak area at 25° C. to a negligible portion of total peak area at 75° C. (FIGS. 2A and B), suggestive of changes in either higher order aggregates or conformational isomers as a function of temperature with a transition temperature somewhere between 65 C and 75 C (FIGS. 2C and 2D).

Since no new peaks appeared when the leading shouder peak disappeared, the total area was compared for the runs made at different temperatures in order to assess if the leading shoulder peak converted to the main peak. Table 2 shows that the total areas from constant load amounts are relatively reproducable from 25° C. to 75° C.; the greatest variability observed was a 24% CV for the 25 μg load, all other load amounts fell within 10% CV across all temperatures tested. Reproducability of total SEC peak area vs run temperature was tested on another occasion (Table 3) to show that a 25 μg load resulted in reproducable total peak area between 25° C., 55° C., and 65° C. (2.3% CV) and the total peak area reproducability between 25° C. and 75° C. run temperatures was slightly more variable (8.1% CV). This data suggests that as the shoulder peak deminishes with increasing temperature, the species of RNA representing the higher molecular weight pre-peak shoulder transitions to co-elute with the main peak. The RNA which at lower temperatures is represented by the pre-peak shoulder may at higher run temperatures shift to the same species as that of the main peak or it may transition to another species that is unresolved under current SEC conditions (and thereby co-elutes with the main peak).

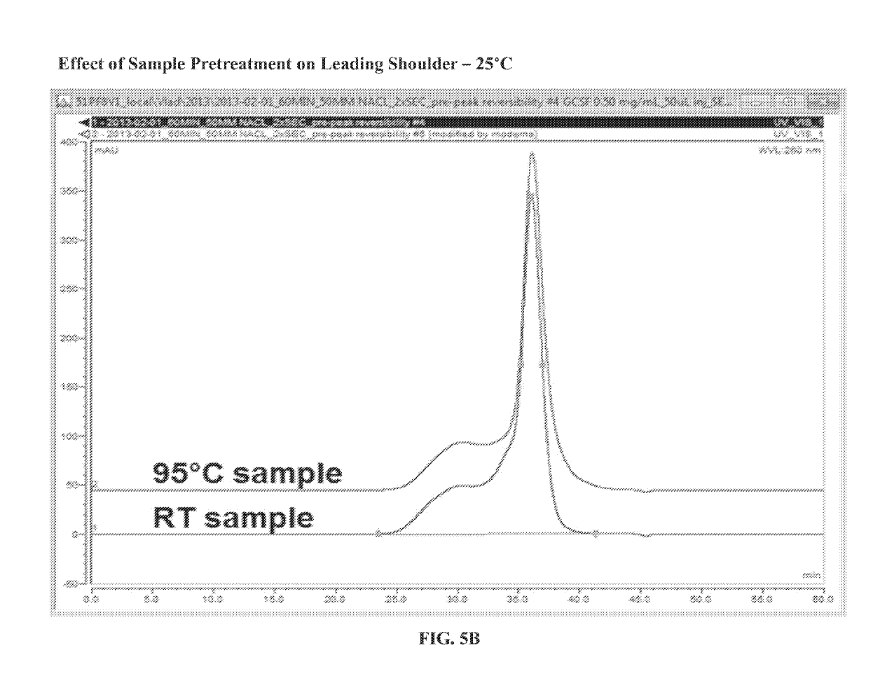

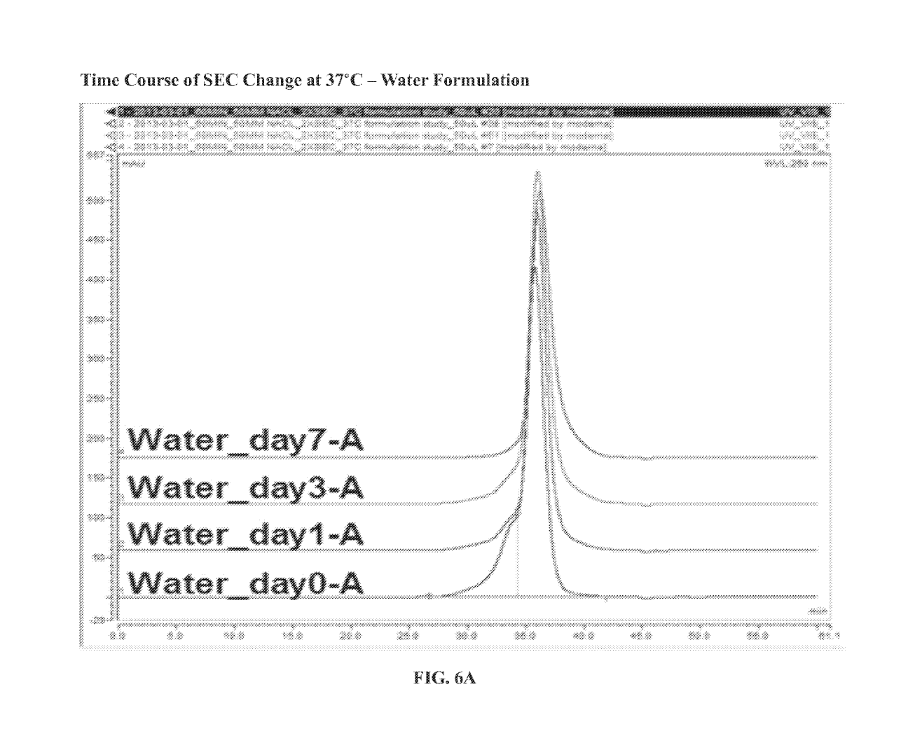

The relative consistency of total peak area regardless of run temperature or elution profile implies that the observed transition of the higher molecular weight pre-peak shoulder may be a reversible event. The reversibility of apparent size was further supported by the finding that sample pre-treatment at 95 C for 5 min, then applying the sample to SEC column at ambient temperature showed the same bimodal profile as not denaturing the sample prior to injection onto the SEC column (FIG. 5B). The determinant of mRNA structural heterogeneity is the continuous exposure to denaturing run conditions as shown in FIG. 5A. Thus, the SEC procedure developed here (comparison of 25 C vs 75 C column temperature) is a useful procedure for differentiating structural variants.

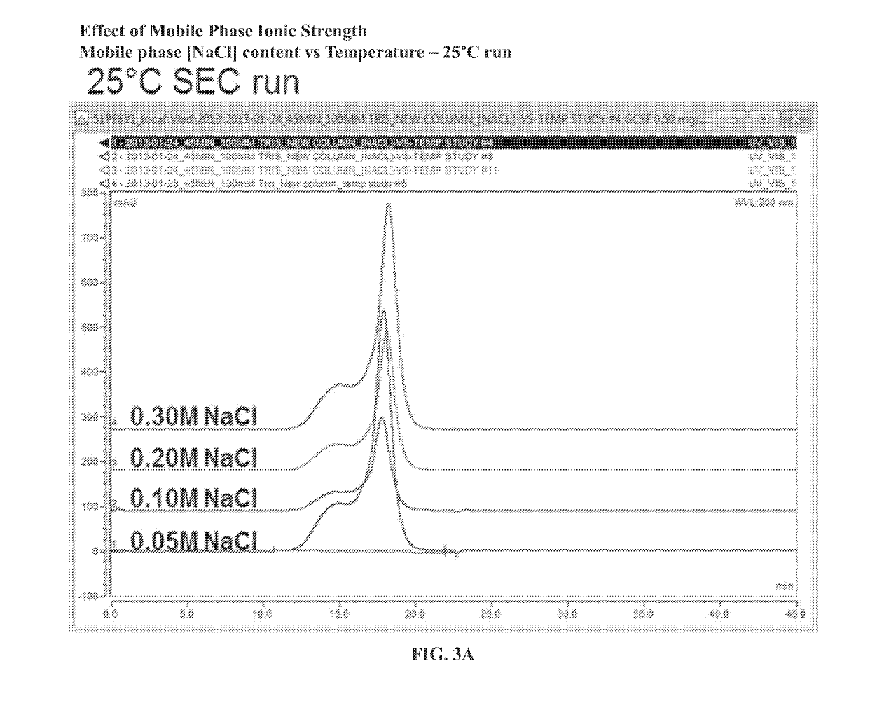

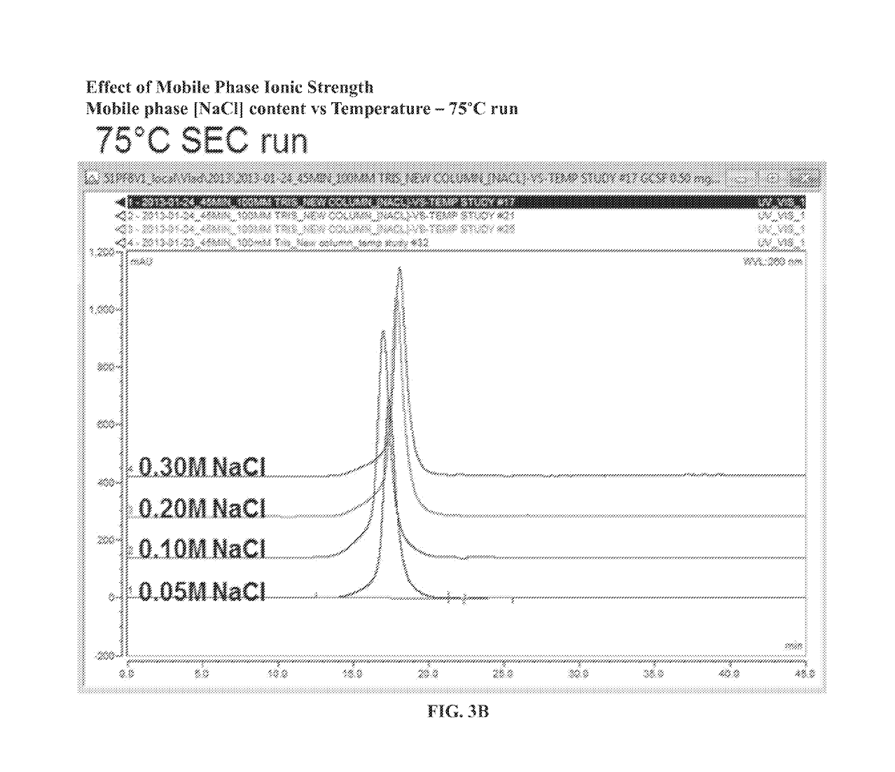

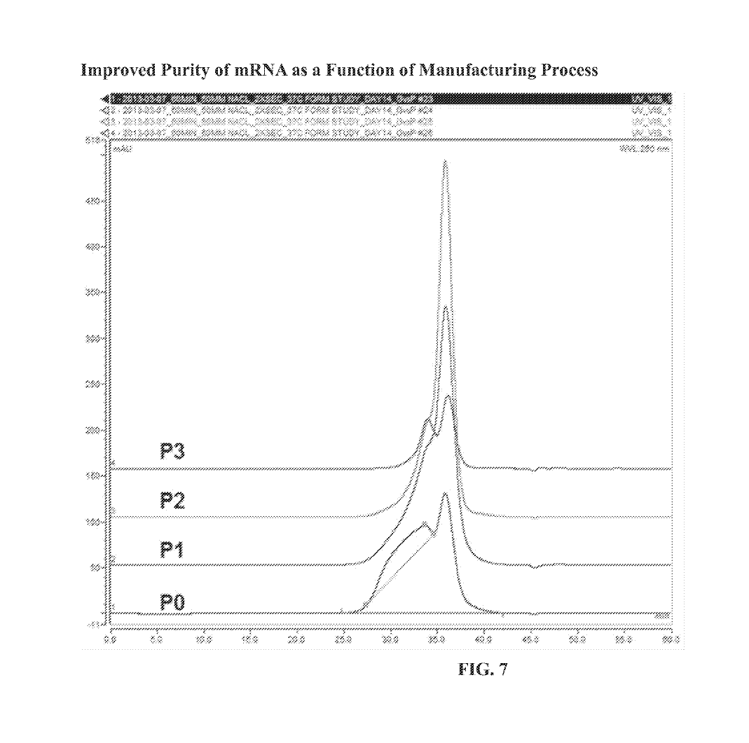

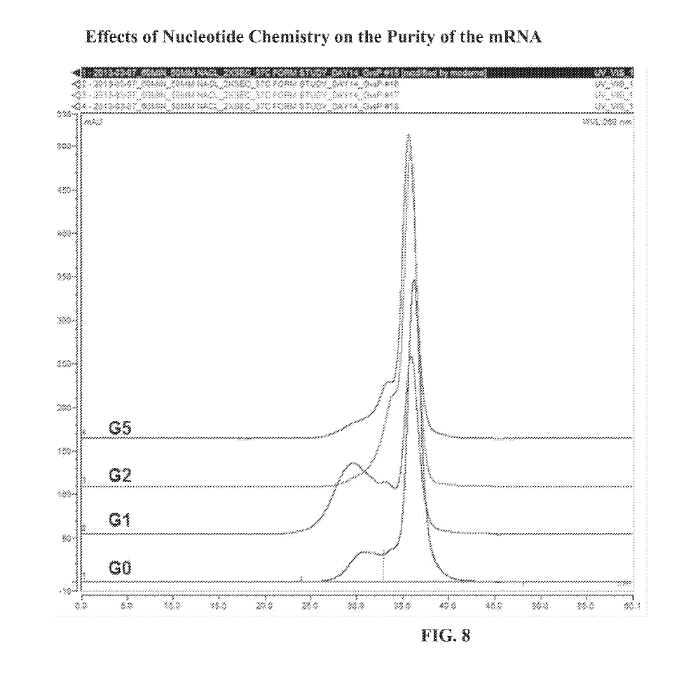

The SEC method was also useful in evaluating the size heterogeneity of different manufacturing processes, where optimization in the process by the use of affinity chromatography (P2 and P3 versus P1) was also corroborated by greater homogeneity as observed by SEC (FIG. 7). Different mobile phase salt concentrations were tested and no significant changes in elution profile was observed at either 25° C. or 75° C. (FIG. 3). There was a very slight shift in retention time toward larger size molecules with 100 mM NaCl in the mobile phase at 75° C. 50 mM NaCl salt concentration was chosen as the nominal concentration because the main elution peak at 75° C. was narrowest under this condition (FIG. 3).

TABLE 2
Reproducibility of Total SEC Peak Area at Different Temperatures

| | Run Temp | | Total Area | Average | |
|---|---|---|---|---|---|
| Sample | (° C.) | Load (uL) | (mAU * min) | Area | % CV |
| GCSF G2 | 25 | 5 | 65.00 | 65.26 | 6% |
| | 35 | 5 | 66.51 | | |
| | 45 | 5 | 60.22 | | |
| | 55 | 5 | 69.31 | | |
| | 25 | 10 | 206.06 | 188.53 | 10% |
| | 35 | 10 | 202.78 | | |
| | 45 | 10 | 198.60 | | |
| | 55 | 10 | 171.45 | | |
| | 65 | 10 | 163.75 | | |
| | 25 | 25 | 621.59 | 590.34 | 6% |
| | 35 | 25 | 621.86 | | |
| | 45 | 25 | 603.08 | | |
| | 55 | 25 | 532.15 | | |
| | 65 | 25 | 573.04 | | |
| | 25 | 50 | 605.84 | 895.20 | 24% |
| | 45 | 50 | 1064.23 | | |
| | 65 | 50 | 1046.55 | | |
| | 75 | 50 | 864.17 | | |

*Note - Some failed runs were removed from the sample set as outliers.

TABLE 3
Reproducibility of Total SEC Peak Area vs Temperatures

| | | | Run | Area | | Total | % CV | % CV |
|---|---|---|---|---|---|---|---|---|
| Sample | | injection | Temp | (mAU * min) | | peak | w/ | (25, 55, |
| No. | Sample Name | (ug) | (° C.) | Pre | Main | area | 25° C. | 65° C.) |
| 5 | GCSF 0.50 mg/mL_25uL inj_SEC buff_25*C_25*C-50mMNaCl-01 | 12.5 | 25 | 152.489 | 453.786 | 606.3 | | 6.3% |
| 9 | GCSF 0.50 mg/mL_25uL inj_SEC buff_25*C_25*C-50mMNaCl-02 | 12.5 | 25 | 160.578 | 466.919 | 627.5 | | |
| 13 | GCSF 0.50 mg/mL_25uL inj_SEC buff_25*C_25*C-50mMNaCl-03 | 12.5 | 25 | 155.802 | 466.729 | 622.5 | | |
| 17 | GCSF 0.50 mg/mL_25uL inj_SEC buff_25*C_55*C-50mMNaCl-01 | 12.5 | 55 | n/a | n/a | n/a | 4.4% | |
| 21 | GCSF 0.50 mg/mL_25uL inj_SEC buff_25*C_55*C-50mMNaCl-02 | 12.5 | 55 | n/a | n/a | n/a | | |
| 25 | GCSF 0.50 mg/mL_25uL inj_SEC buff_25*C_55*C-50mMNaCl-03 | 12.5 | 55 | 137.238 | 431.086 | 568.3 | | |
| 29 | GCSF 0.50 mg/mL_25uL inj_SEC buff_25*C_65*C-50mMNaCl-01 | 12.5 | 65 | | 524.557 | 524.6 | 6.7% | |
| 33 | GCSF 0.50 mg/mL_25uL inj_SEC buff_25*C_65*C-50mMNaCl-02 | 12.5 | 65 | | 560.772 | 560.8 | | |
| 37 | GCSF 0.50 mg/mL_25uL inj_SEC buff_25*C_65*C-50mMNaCl-03 | 12.5 | 65 | | 588.647 | 588.6 | | |
| 41 | GCSF 0.50 mg/mL_25uL inj_SEC buff_25*C_75*C-50mMNaCl-01 | 12.5 | 75 | | 452.012 | 452.0 | 15.5% | |
| 45 | GCSF 0.50 mg/mL_25uL inj_SEC buff_25*C_75*C-50mMNaCl-02 | 12.5 | 75 | | 472.023 | 472.0 | | |
| 49 | GCSF 0.50 mg/mL_25uL inj_SEC buff_25*C_75*C-50mMNaCl-03 | 12.5 | 75 | | 476.044 | 476.0 | | |
| 6 | GCSF 0.50 mg/mL_50uL inj_SEC buff_25*C_25*C-50mMNaCl-01 | 25.0 | 25 | 278.457 | 830.894 | 1109.4 | | 2.3% |
| 10 | GCSF 0.50 mg/mL_50uL inj_SEC buff_25*C_25*C-50mMNaCl-02 | 25.0 | 25 | n/a | n/a | n/a | | |
| 14 | GCSF 0.50 mg/mL_50uL inj_SEC buff_25*C_25*C-50mMNaCl-03 | 25.0 | 25 | 286.642 | 832.776 | 1119.4 | | |
| 18 | GCSF 0.50 mg/mL_50uL inj_SEC buff_25*C_55*C-50mMNaCl-01 | 25.0 | 55 | n/a | n/a | n/a | 2.1% | |
| 22 | GCSF 0.50 mg/mL_50uL inj_SEC buff_25*C_55*C-50mMNaCl-02 | 25.0 | 55 | n/a | n/a | n/a | | |
| 26 | GCSF 0.50 mg/mL_50uL inj_SEC buff_25*C_55*C-50mMNaCl-03 | 25.0 | 55 | 269.483 | 806.281 | 1075.8 | | |
| 30 | GCSF 0.50 mg/mL_50uL inj_SEC buff_25*C_65*C-50mMNaCl-01 | 25.0 | 65 | | 1054.758 | 1054.8 | 2.5% | |
| 34 | GCSF 0.50 mg/mL_50uL inj_SEC buff_25*C_65*C-50mMNaCl-02 | 25.0 | 65 | | 1071.735 | 1071.7 | | |
| 38 | GCSF 0.50 mg/mL_50uL inj_SEC buff_25*C_65*C-50mMNaCl-03 | 25.0 | 65 | | 1078.576 | 1078.6 | | |
| 42 | GCSF 0.50 mg/mL_50uL inj_SEC buff_25*C_75*C-50mMNaCl-01 | 25.0 | 75 | | 926.625 | 926.6 | 8.1% | |
| 46 | GCSF 0.50 mg/mL_50uL inj_SEC buff_25*C_75*C-50mMNaCl-02 | 25.0 | 75 | | 956.658 | 956.7 | | |
| 50 | GCSF 0.50 mg/mL_50uL inj_SEC buff_25*C_75*C-50mMNaCl-03 | 25.0 | 75 | | 962.622 | 962.6 | | |
| 51 | GCSF 0.50 mg/mL_50uL inj_SEC buff_25*C_75*C-50mMNaCl-03 | 25.0 | 75 | | 1015.841 | 1015.8 | | |

Effect of Mobile Phase Ionic Strength—FIG. 3

The 50 mM NaCl containing mobile phase corresponded to the narrowest peak shape at 75° C., thus 50 mM NaCl was chosen as the nominal salt concentration for the SEC mobile phase.

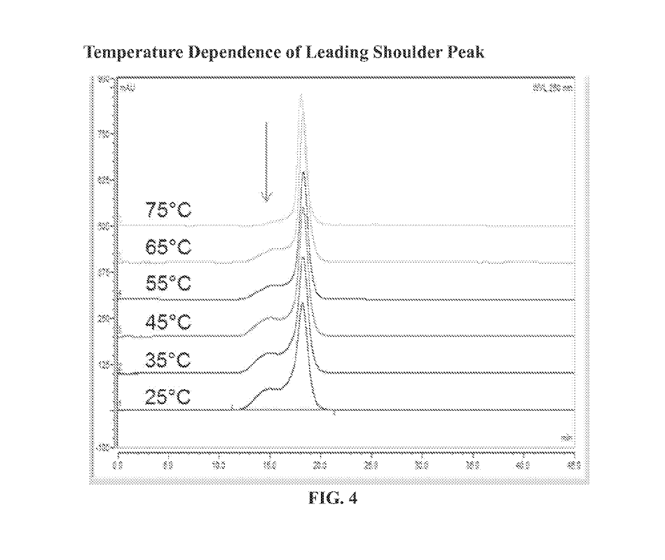

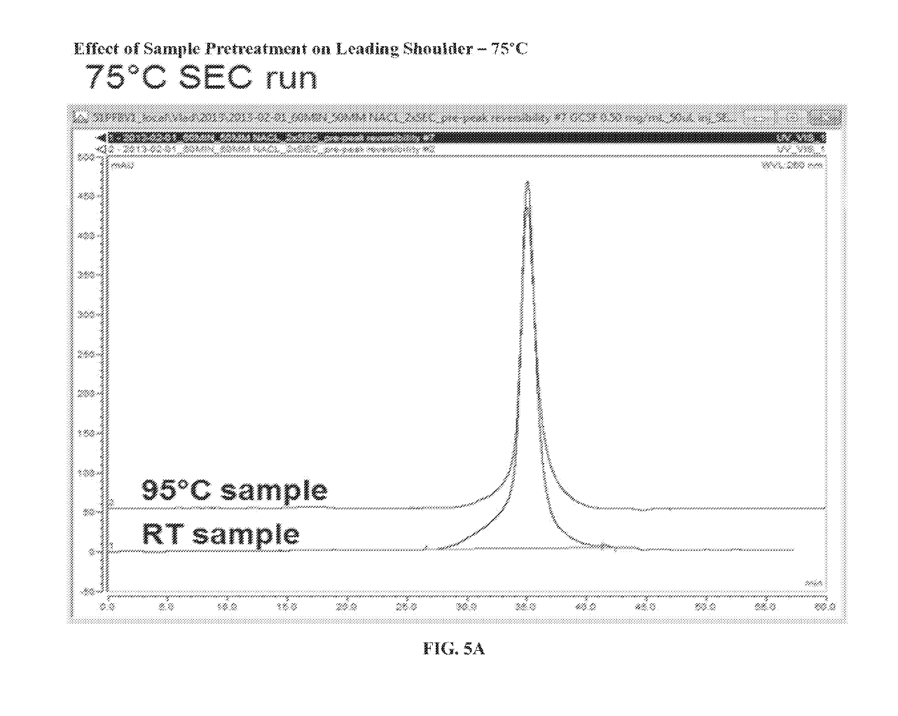

Temperature Dependence of SEC—FIG. 4

FIG. 4 shows the SEC results from repeat injection of the same sample (0.5 mg/mL GCSF) at increasing column temperatures. The shoulder peak (arrow) is reduced significantly between 65° C. and 75° C.

Reversability of Leading Shoulder Peak—FIG. 5

Two samples of 0.5 mg/mL GCSF were prepared by dilution into SEC mobile phase (Table 1). One sample was denatured at 95° C. for 5 min and then allowed to equilibrate to room temperature over at least 1 hour, the other was stored at room temperature for at least 3 hrs before being injected onto the column that was held at either 25° C. or 75° C.

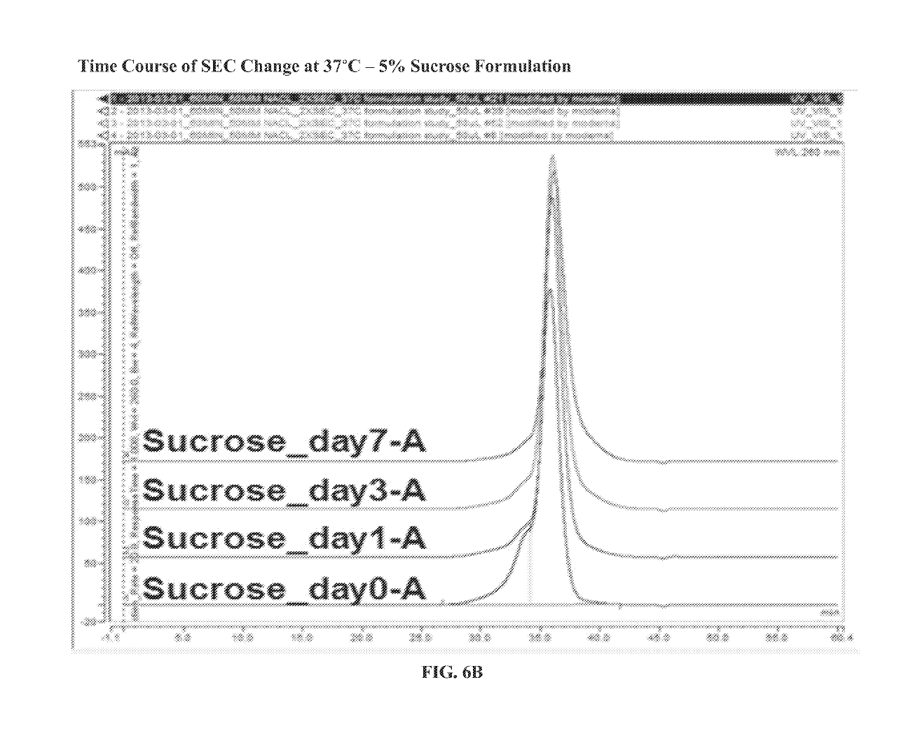

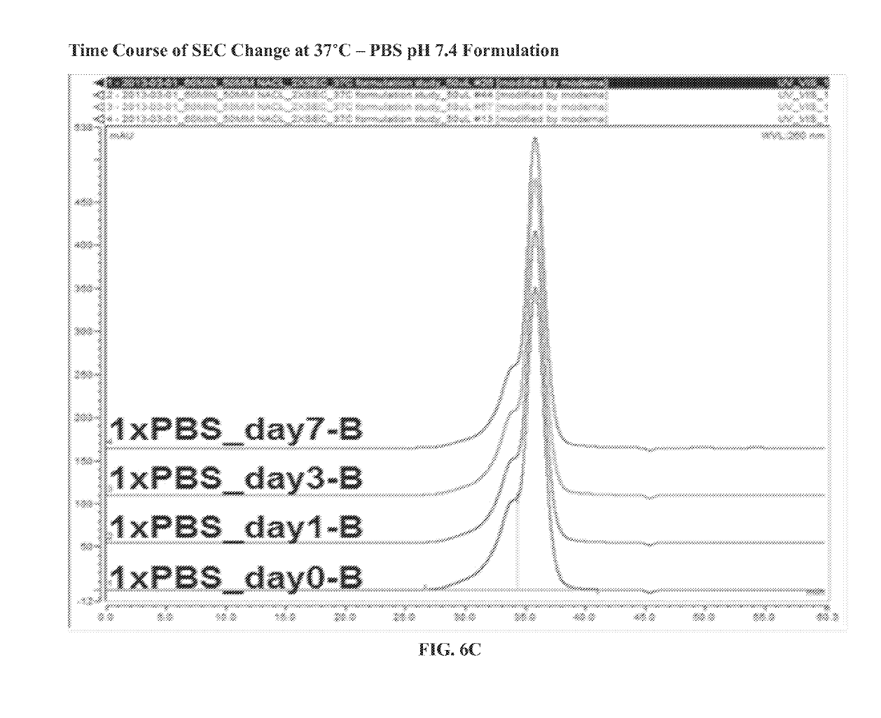

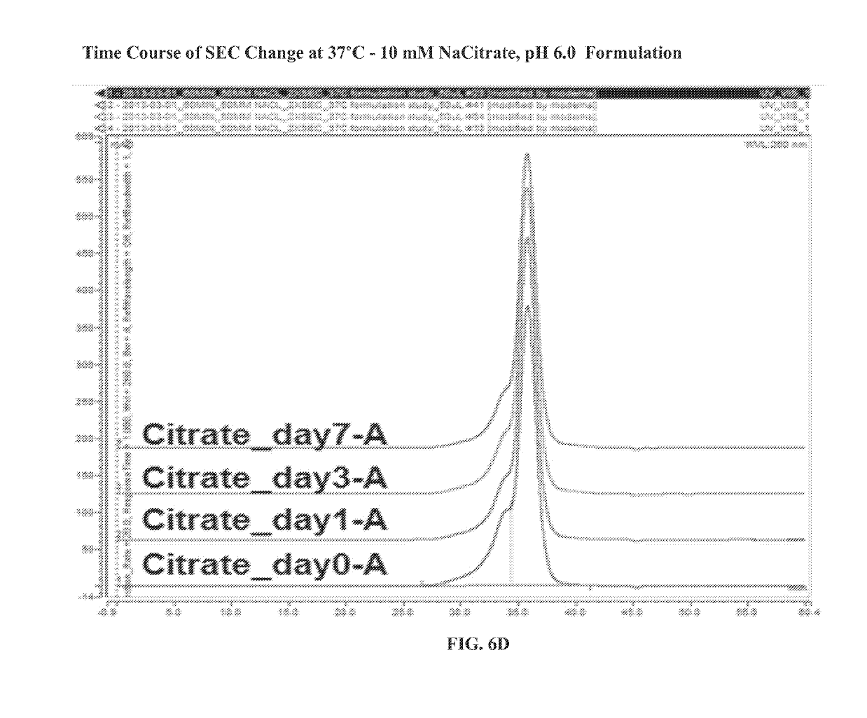

Stress Stability and Formulation Study—FIG. 6

The effect of four formulation compositions on the thermal stability of mRNA at 37° C. was evaluated using a non-denaturing SEC conditions (25° C.). As shown in FIG. 6, water and sucrose formulations lost the leading shoulder peak during the 7 days of stability study, whereas buffered formulations PBS and Citrate showed no significant change in the profile. Since there was no change in pH during the time course of the study, this stabilization by PBS and citrate might be due to the chelating ability of these buffers. mRNA is known to breakdown in the presence of small amounts of divalent cations, Mg specifically [ref], and phosphate and citrate would chelate these ions.

Improved Purity of mRNA as a Function of Manufacturing Process—FIG. 7

The earlier eluting peaks are likely due to tail-less material in the P0 and P1 processes, where these species are removed by the oligo dT process.

### Example 2

### Analysis of Purity, Heterogeneity, Impurities, and Stability of mRNA by RP-HPLC

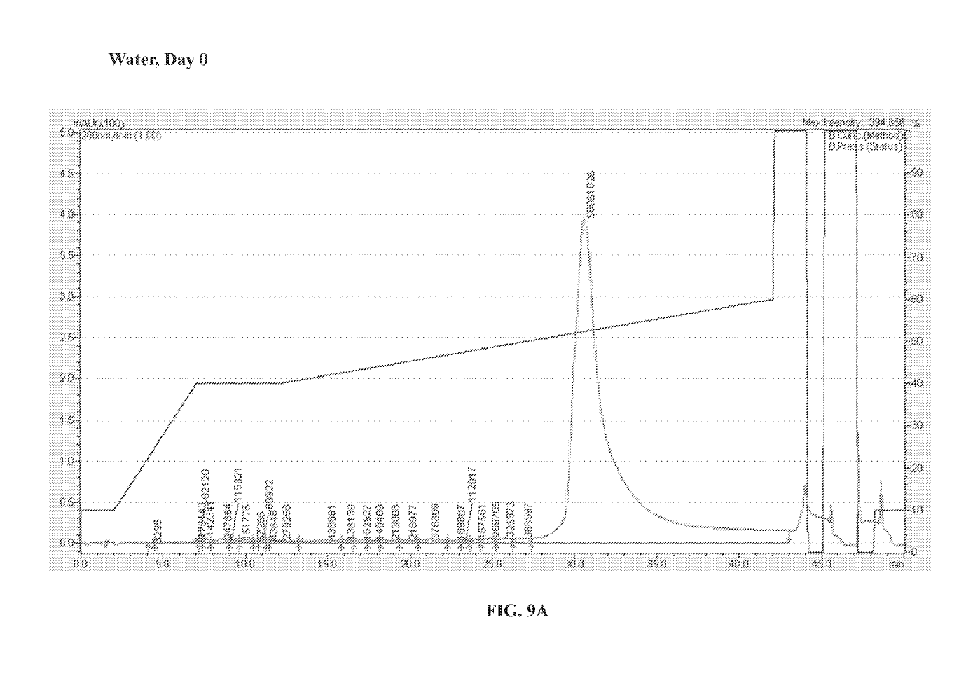

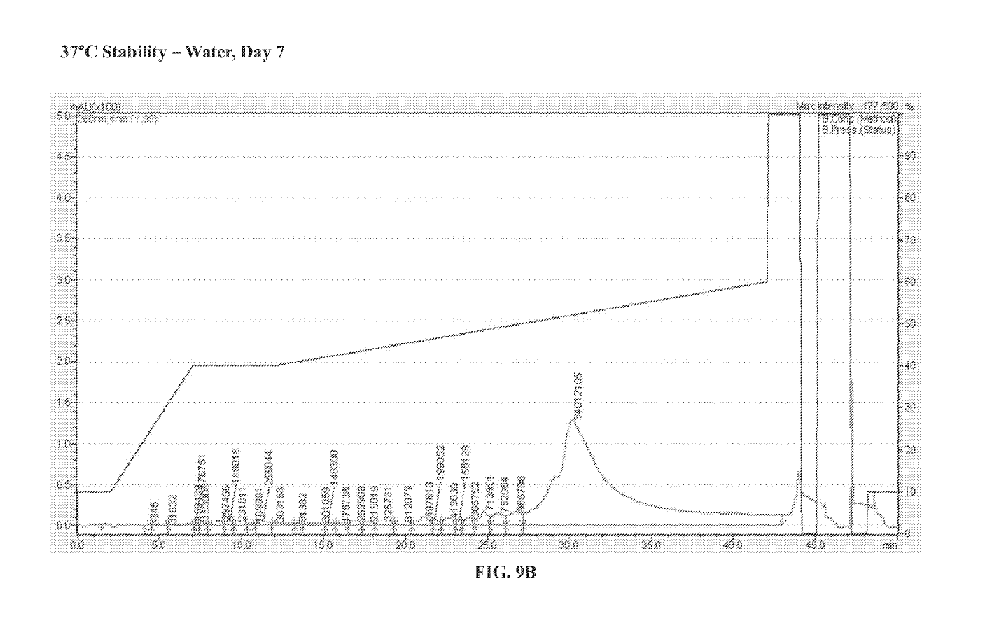

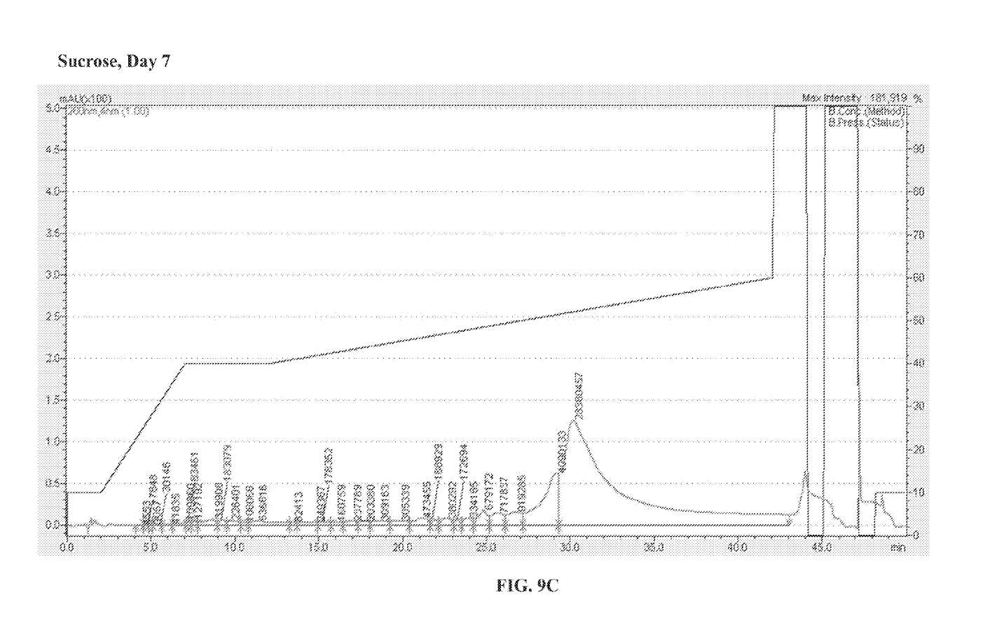

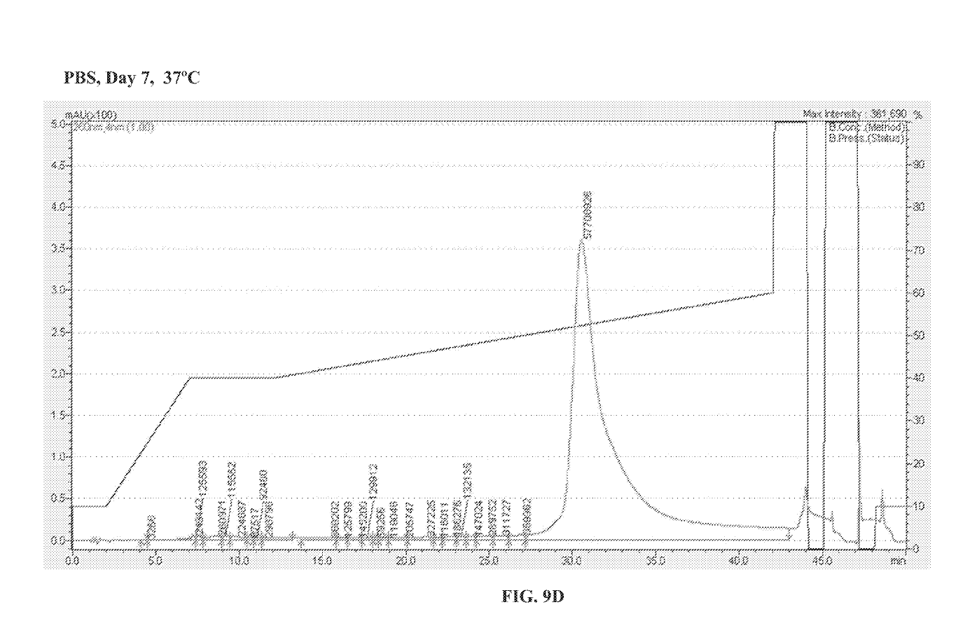

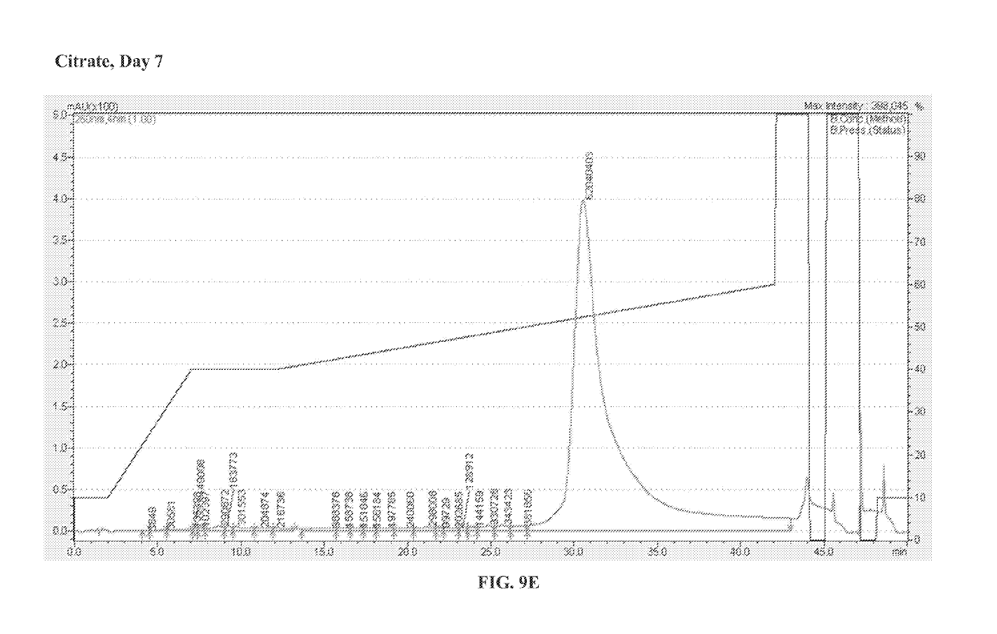

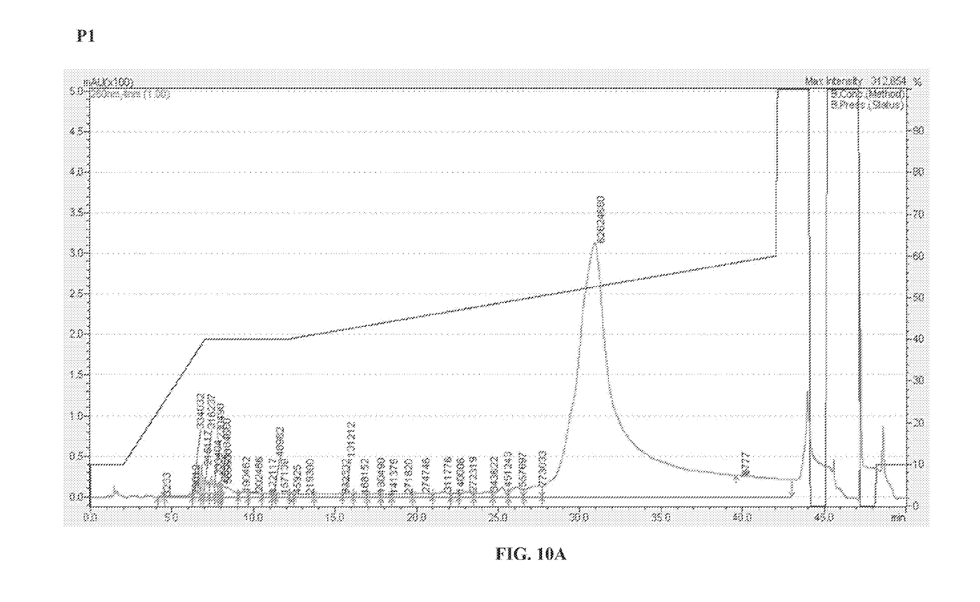

A RP-HPLC method was developed to evaluate mRNA quality. FIG. 9 shows that the method is stability indicating, as it shows greater degradation in unbuffered formulations than buffered one, consistent with the SEC observation (FIG. 9). The earlier eluting shoulder peak in water (FIG. 9B) and sucrose (FIG. 9C) is suggestive of fragmentation during storage at 37 C. The presence of buffers, which could also chelate divalent cations, seems to enhance stability towards fragmentation (FIGS. 9D-E).

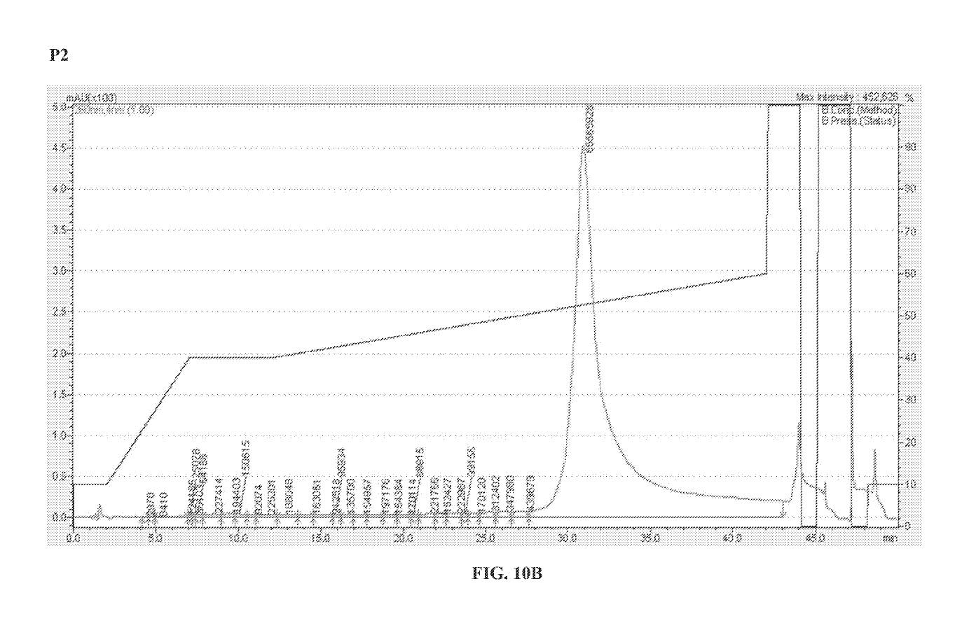

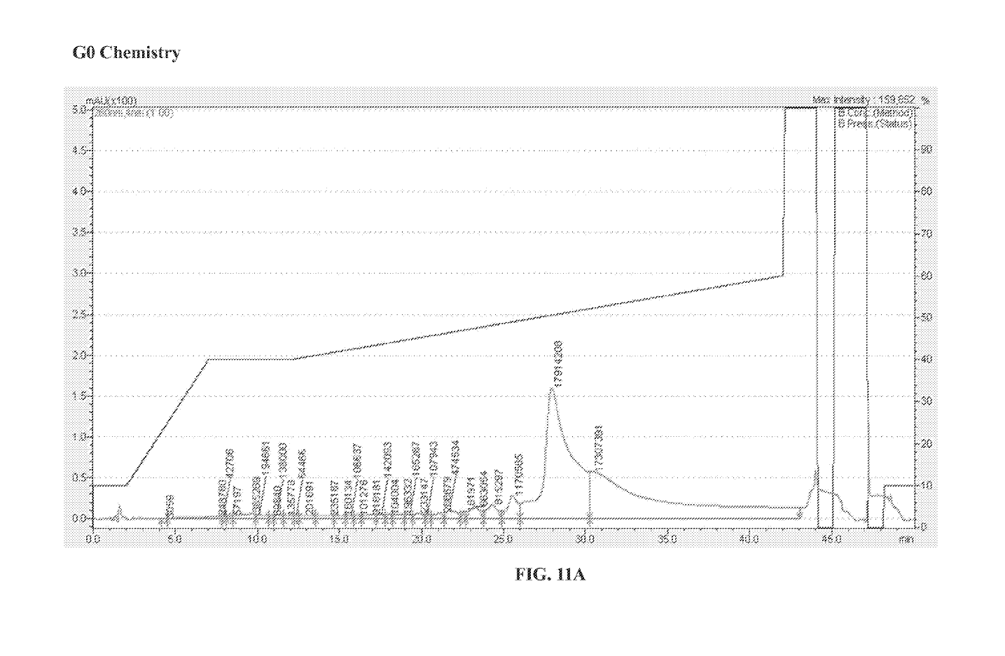

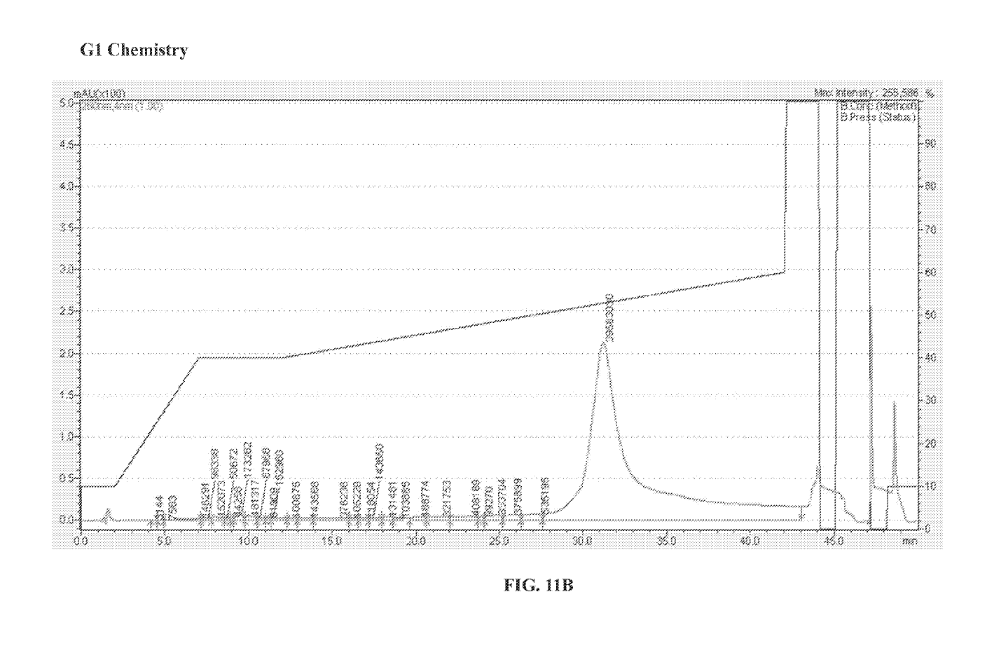

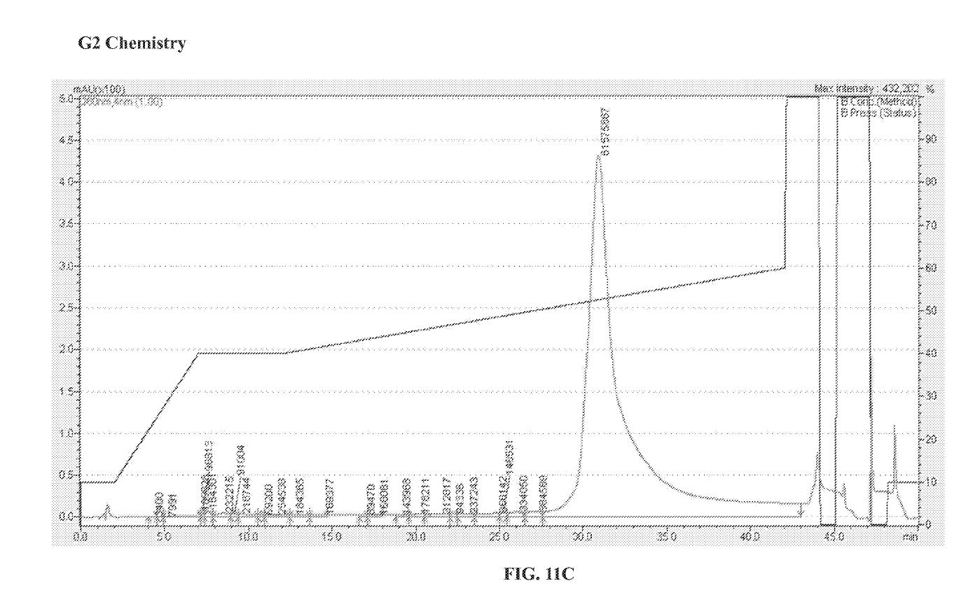

Comparison of the RP-HPLC profile of mRNA prepared by an earlier manufacturing process (P1) and the oligo dT purified mRNA (P2) shows the broader peak and increased tailing at both sides of the peak (FIG. 10), indicative of greater heterogeneity in P1 due different lengths of mRNA and other impurities. The minor inflection at the leading edge (FIG. 10A, arrow) is likely due to the tail-less species.

This method was also able to detect differences in mRNA composed of different chemistries.

TABLE 4
Reverse Phase LC-UV Method Summary

| | |
|---|---|
| Column | Waters XBridge C18, 2.1 × 50 mm |
| Column Heater | 35° C. |
| Mobile Phase A | 100 mM TEAA, pH 7.0 |
| Mobile Phase B | 100 mM TEAA, 25% Acetonitrile, pH 7.0 |
| Flow Rate | 0.50 mL/min |
| Injection Volume | 50 μL |
| Detection Wavelength | 260 nm |
| Total Run Time | 55 min |

TABLE 5
Reversed Phase LC-UV Run Conditions

| Time (min) | Flow (mL/min) | % A | % B |
|---|---|---|---|
| initial | 0.50 | 80 | 20 |
| 1.0 | 0.50 | 80 | 20 |
| 7.0 | 0.50 | 60 | 40 |
| 12.0 | 0.50 | 60 | 40 |
| 42.0 | 0.50 | 45 | 55 |
| 42.1 | 0.50 | 0 | 100 |
| 44.0 | 0.50 | 0 | 100 |
| 44.1 | 0.50 | 100 | 0 |
| 45.0 | 0.50 | 100 | 0 |
| 45.1 | 0.50 | 0 | 100 |
| 47.0 | 0.50 | 0 | 100 |
| 47.1 | 0.50 | 100 | 0 |
| 48.0 | 0.50 | 100 | 0 |
| 48.1 | 0.50 | 80 | 20 |
| 50.0 | 0.50 | 80 | 20 |

RP-HPLC Profile of mRNA Prepared by Different Manufacturing Processes—FIG. 10

The broader peak width of the P1 process and greater tailing towards later eluting as well as a poorly resolving inflection at the leading edge where species lacking polyA are expected to elute is indicative of greater heterogeneity associated with different length mRNA and other impurities.

Size Exclusion Chromatography with On-Line Light Scattering Detection

The actual diameter is definitively measured by multi-angle laser light scattering either in batch mode (such as Wyatt's HELIOS system) or on-line with the SEC (such as Wyatt's MiniDawn). For on-line analysis, the light scattering detector and refractive index detector are placed after the SEC column chromatography elution along with UV detector, enabling calculation of the weight averaged molecular weight of mRNA species. The dn/dc (change in refractive index as a function of mRNA) is measured, and used for calculating the MW of the observed species on the SEC column according to well-established equations. By comparing the calculated MW with the observed retention time on SEC, this analysis provides definitive evidence whether the shoulder observed at ambient temperature in mRNA preparations is a conformational isoform (as it would have the same MW as the main peak) or aggregated species with much larger MW than the mRNA in the main peak. The method also confirms the MW of the main peak and might show size heterogeneity even in the main peak within the resolution of the system, which is approximately 2-3 kDa.

Characterization of mRNA Species Observed by SEC

For better understanding of the nature of SEC separation, the shoulder peak observed in large mRNA preparations at ambient temperature is collected until adequate amount of the peak is available for subsequent characterization by AEX, RP-HPLC, oligonucleotide mapping followed by UV and LC-MS characterization. Additionally, the relative thermal stability of the shoulder peak and the main peak is evaluated to support the observation that the shoulder peak appears to be a conformational isomer of the main peak.

### Preparative Size Exclusion Chromatography for Purification

SEC has been previously utilized in small-scale purification of in vitro transcribed RNAs of sizes typically less than several hundred nucleotides at lab scale. Short RNA transcripts (e.g., less 400 nucleotides) were separated from plasmid DNA templates, nucleotide triphosphates, and other short abort sequences generated during transcription. These results were achieved under non-denaturing conditions.

SEC preparative purification methods of the present invention can separate these aforementioned impurities, but can also be used to remove hybridized nucleic acid impurities as well as multimeric RNA species. This method allows for the purification of large RNA transcripts and chemically modified RNA transcripts. Hybridized nucleic acid contaminants may be generated through the RNA manufacturing process. Common examples of hybridized impurities include:
1) Double stranded RNA (dsRNA) of various length: These may originate from aberrant RNA transcripts formed during the in vitro transcription
2) RNA:DNA hybrids: The most probable source would be generated from incomplete removal of fragments of DNA template that hybridize to complementary sequences of RNA following DNase digestion.
3) RNA:DNA or RNA:RNA (including modified nucleotides): the source of DNA could be leaching of the affinity ligand from oligonucleotide-based resins.

These species have the potential to activate the innate immune response and therefore their level must be minimized and controlled during manufacture of mRNA-based therapeutics. Use of denaturing conditions and/or chaotropic agents in the SEC mobile phase facilitates the dissociation the hybridized impurity from the RNA that can then be easily resolved from the RNA. The preparative SEC purification step can then generate RNA free of hybridized nucleic acid impurities.

### Other Methodologies to Assess Structural Heterogeneity and Stability

Other methods can also be used to assess structural heterogeneity and stability. These methods include obtaining the RNA transcript, such as a large RNA transcript or one that is chemically modified, and characterizing the RNA transcript using a procedure, such as chip-based capillary electrophoresis, agarose gel electrophoresis, analytical ultracentrifugation, and field flow fractionation.

Chip-Based Capillary Electrophoresis

Chip-based capillary electrophoresis (e.g. with the AGILENT 2100 BIOANALYZER™) can be used as a as a rapid and routine method for monitoring RNA transcript integrity and its size distribution as a function of the manufacturing process and stability/handling. The separation is based on hydrodynamic size and charge, and is affected by the nucleotide length and folded structure of the RNA transcript. In one embodiment, the method includes delivering the sample into a channel of a chip with an electrolyte medium and applying an electric field to the chip that causes the RNA transcript and the impurities migrate through the channel. The RNA transcript has a different electrophoretic mobility than the impurities such that the RNA transcript migrates through the channel at rate that is different from a rate at which the impurities migrate through the channel. The electrophoretic mobility of the RNA transcript is proportional to an ionic charge the RNA transcript and inversely proportional to frictional forces in the electrolyte medium. The method also includes collecting from the chip the sample comprising the RNA transcript and one or more separate portions of the sample comprising the impurities. In addition, the method includes characterizing an aspect of at least one of the portion of the sample comprising the RNA transcript and the one or more separate portions of the sample comprising the impurities. The characterizing can include, for example, quantifying charge variants.

Agarose Gel Electrophoresis

RNA transcript topology and apparent (hydrodynamic) size can also be analyzed by agarose gel electrophoresis, for example on a 1.2% agarose gel and using PicoGreen binding with fluorescence detection. The agarose gel method gives a more quantitative, but less resolving, measure of size distribution.

Analytical Ultracentrifugation (AUC)

Analytical ultracentrifugation (AUC) is a solution phase method for measuring molecular weight distribution, without the potential artifacts that could be introduced by matrix (resin or gel) interaction in the SEC, agarose, or other methods. Both equilibrium AUC and sedimentation ultracentrifugation are used, and the latter provides sedimentation coefficients that are related to both size and shape of the RNA transcript. A BECKMAN™ analytical ultracentrifuge equipped with a scanning UV/visible optics is used for analysis of the RNA transcript.

Field Flow Fractionation

Another solution phase method for assessing hydrodynamic size distribution is field flow fractionation.

### General Measurements

For all the above technologies, the following measurements are made:
1. as a function of RNA transcript concentration to assess concentration dependent self-association and reversibility
2. as a function of temperature to correlate the data with the bimodal distribution observed by SEC. The thermal data are compared with differential scanning calorimetric (DSC) analysis, which characterizes the thermal stability of macromolecules by measuring differential heat corresponding to structural changes that occur in the sample. Large RNA transcripts are expected to show several transitions reflecting the sequence-dependent micro-structures (e.g., hairpins, loops, double stranded regions, stacks, or higher order structures) that can in turn affect the elution on SEC. DSC measurements are performed using a Microcal capillary DSC or equivalent. Another common method for detecting nucleotide structural transitions reflective of secondary structure is UV melting profile since UV absorption is sensitive to changes in base pair interactions. The UV absorption spectrum of mRNA is evaluated as a function of temperature and transitions compared with the temperature dependent transitions on SEC.
3. Stability samples are also evaluated by the various orthogonal methods described above to compare with the altered SEC (FIG. 7) and RP-HPLC (FIG. 10) profiles.

The collective analyses provide a comprehensive view of the size and structural heterogeneity and stability of large mRNAs.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### REFERENCES CITED

1. Ketterer L, Von Der Mulbe F, Reidel, L, Mutzke, T. U.S. Pat. No. 8,383,340.
2. Lukaysky P, Puglisi J. Large-scale preparation and purification of polyacrylamide-free RNA oligonucleotides. RNA 2004, 10(5): 889-893.
3. Kim I, McKenna S, Puglisi E, Puglisi J. Rapid purification of RNAs using fast performance liquid chromatography (FPLC). RNA 2007, 13:289-294.
4. Graeve L, Goemann W, Foldi P, Kruppa J. Fractionation of biologically active messenger RNAS by HPLC gel filtration. Biochemical and Biophysical Research Communications. 1982, 107(4):1559-156.
5. Rott R, Zipor G, Portnoy V, Liveanu V, Schuster G. RNA polyadenylation and degradation in cyanobacteria are similar to the chloroplast but different from Escherichia coli. J. Biol. Chem. 2003, 278(18):15771-15777.
6. Gillar et al., Ion-pair reversed-phase high-performance liquid chromatography analysis of oligonucleotides: Retention prediction. J. Chrom. 2002, 958(1):167-182
7. Azarani A., Hecker K H. RNA analysis by ion-pair reversed-phase high performance liquid chromatography. Nucleic Acids Res. 2001, 29(2): e7.

1. A method for characterizing an aspect of a sample comprising a ribonucleic acid (RNA) transcript and impurities, the method comprising:
delivering the sample across a reversed phase that is a stationary phase, the sample delivered with at least one mobile phase, wherein the RNA transcript and the impurities in the sample interact with the reversed phase to different degrees such that the RNA transcript elutes through the reversed phase at rate that is different from a rate at which each of the impurities elute through the reversed phase, wherein the RNA transcript is 300 to 10,000 nucleotides in length or is chemically modified;
eluting from the reversed phase a portion of the sample comprising the RNA transcript and one or more separate portions of the sample comprising the impurities; and
characterizing an aspect of the portion of the sample comprising the RNA transcript or the portions of the sample comprising the impurities.

2. The method of claim 1, wherein the RNA transcript is 700 to 3,000 nucleotides in length.

3. The method of claim 1, wherein the RNA transcript is 800 to 2,000 nucleotides in length.

4. The method of claim 1, wherein the RNA transcript is a full length RNA transcript.

5. The method of claim 1, wherein the RNA transcript is chemically modified.

6. The method of claim 1, wherein the at least one mobile phase comprises a non-methanol mobile phase.

7. The method of claim 1, wherein the at least one mobile phase comprises a non-acetonitrile mobile phase.

8. The method of claim 1, wherein the at least one mobile phase comprises methanol.

9. The method of claim 1, wherein the at least one mobile phase comprises acetonitrile.

10. The method of claim 1, wherein at least one of the steps is performed under denaturing conditions.

11. The method of claim 10, wherein the denaturing conditions comprise an elevated temperature sufficient to denature intramolecular hydrogen bonds.

12. The method of claim 10, wherein the denaturing conditions comprise the presence of a chaotropic agent.

13. The method of claim 1, further comprising pre-incubating the sample at an elevated temperature sufficient to denature intramolecular hydrogen bonds.

14. The method of claim 1, wherein the method is a column chromatography method in which the sample is delivered through a column and wherein the column is run at an elevated temperature sufficient to denature intramolecular hydrogen bonds.

15. The method of claim 1, wherein characterizing an aspect comprises quantifying the impurities eluted in the one or more separate portions.

16. The method of claim 1, wherein characterizing an aspect comprises detecting heterogeneity of the portion of the sample comprising the RNA transcript.

17. The method of claim 1, wherein the RNA transcript is the product of in vitro transcription using a non-amplified DNA template.

18. A method for characterizing an aspect of a sample comprising a ribonucleic acid (RNA) transcript and impurities, the method comprising:
delivering the sample across a stationary phase comprising a plurality of pores, the sample delivered with at least one mobile phase, wherein the RNA transcript is a different size than the impurities and wherein the plurality of pores are of a size that permits the RNA transcript to elute through the stationary phase at rate that is different from a rate at which the impurities elute through the stationary phase, wherein the RNA transcript is 300 to 10,000 nucleotides in length or is chemically modified;
eluting from the stationary phase at least one of the portion of the sample comprising the RNA transcript and one or more separate portions of the sample comprising the impurities; and
characterizing an aspect of the portion of the sample comprising the RNA transcript and the one or more separate portions of the sample comprising the impurities.

19. The method of claim 18, wherein the RNA transcript is 700 to 3,000 nucleotides in length.

20. The method of claim 18, wherein the RNA transcript is 800 to 2,000 nucleotides or base pairs in length.

21. The method of claim 18, wherein the RNA transcript is a full length RNA transcript.

22. The method of claim 18, wherein the RNA transcript is chemically modified.

23. The method of claim 18, where at least one of the steps is performed under denaturing conditions.

24. The method of claim 23, wherein the denaturing conditions comprise an elevated temperature sufficient to denature intramolecular hydrogen bonds.

25. The method of claim 23, wherein the denaturing conditions comprise the presence of a chaotropic agent.

26. The method of claim 23, wherein the chaotropic agent is selected from a group consisting of: perchlorate salts, guanidinium salts, and urea.

27. The method of claim 18, further comprising pre-incubating the sample at an elevated temperature sufficient to denature intramolecular hydrogen bonds.

28. The method of claim 18, wherein the method is a column chromatography method in which the sample is delivered through a column and wherein the column is run at an elevated temperature sufficient to denature intramolecular hydrogen bonds.

29. The method of claim 18, where at least one of the steps is performed under partially denaturing conditions.

30. The method of claim 18, where at least one of the steps is performed under non-denaturing conditions.

31. The method of claim 18, wherein the method comprises using size exclusion chromatography under denaturing and non-denaturing conditions to discover conformational information about the RNA transcript.

32. The method of claim 18, wherein the impurities comprise hybridized nucleic acid impurities from RNA transcript preparations.

33. The method of claim 32, wherein hybridized nucleic acid impurities comprise double stranded RNA.

34. The method of claim 32, wherein hybridized nucleic acid impurities comprise RNA:DNA hybrids.

35. The method of claim 32, wherein hybridized nucleic acid impurities comprise DNA or RNA that is an affinity ligand leached from oligonucleotide-based resins.

36. The method of claim 18, wherein the stationary phase is selected from a group consisting of: poly styrene divinylbenzene, polymethacrylate, crosslinked agarose, allyl dextran with N-N-bis acrylamide, silica, dextran, polyacrylamide, hydrophilic media, and hydrophobic media.

37. The method of claim 18, wherein characterizing an aspect comprises differentiating structural isoforms of the RNA transcript.

38. The method of claim 18, wherein the RNA transcript is the product of in vitro transcription using a non-amplified DNA template.

39. The method of claim 18, wherein the characterizing is performed with on-line light scattering.

40. The method of claim 39, wherein the on-line light scattering is performed with a light scattering detector and a refractive index detector placed for detection after the eluting step, and with a UV detector.

41. The method of claim 40, wherein the change in refractive index as a function of the RNA transcript is measured and used for calculating a molecular weight of the portion of the sample comprising the RNA transcript or the one or more separate portions of the sample comprising the impurities.

42. The method of claim 41, wherein the molecular weight is compared with a retention time on the stationary phase to determine whether the portion or the one or more separate portions comprise a conformational isoforms or an aggregated species with a larger molecular weight.

43. The method of claim 18, wherein the characterizing is performed by anion exchange chromatography, reversed phase-high performance liquid chromatography, or oligonucleotide mapping followed by UV and LC-MS characterization.

44. The method of claim 18, wherein differential scanning calorimetric (DSC) analysis is used to characterize thermal stability of the sample by measuring differential heat corresponding to structural changes that occur in the sample.

45. The method of claim 18, wherein UV melting profile of the RNA transcript is used for detecting nucleotide structural transitions reflective of secondary structure of the RNA transcript.

46. A method for purifying a sample comprising a ribonucleic acid (RNA) transcript and impurities, the method comprising:
delivering the sample across a stationary phase comprising a plurality of pores, the sample delivered with at least one mobile phase, wherein the RNA transcript is a different size than the impurities and wherein the plurality of pores are of a size that permits the RNA transcript to elute through the stationary phase at rate that is different from a rate at which the impurities elute through the stationary phase such that the impurities in the sample pass through the stationary phase, wherein the RNA transcript is 300 to 10,000 nucleotides in length or is chemically modified; and
eluting from the stationary phase a purified sample comprising the RNA transcript.

47. The method of claim 46, wherein the RNA transcript is 700 to 3,000 nucleotides in length.

48. The method of claim 46, wherein the RNA transcript is 800 to 2,000 nucleotides or base pairs in length.

49. The method of claim 46, wherein the RNA transcript is a full length RNA transcript.

50. The method of claim 46, wherein the RNA transcript is chemically modified.

51. The method of claim 46, where at least one of the steps is performed under denaturing conditions.

52. The method of claim 51, wherein the denaturing conditions comprise an elevated temperature sufficient to denature intramolecular hydrogen bonds.

53. The method of claim 51, wherein the denaturing conditions comprise the presence of a chaotropic agent.

54. The method of claim 53, wherein the chaotropic agent is selected from a group consisting of: perchlorate salts, guanidinium salts, and urea.

55. The method of claim 46, further comprising pre-incubating the sample at an elevated temperature sufficient to denature intramolecular hydrogen bonds.

56. The method of claim 46, wherein the method is a column chromatography method in which the sample is delivered through a column and wherein the column is run at an elevated temperature sufficient to denature intramolecular hydrogen bonds.

57. The method of claim 46, wherein the method is useable for large scale purification of RNA transcripts.

58. The method of claim 46, wherein the impurities comprise hybridized nucleic acid impurities from RNA transcript preparations.

59. The method of claim 58, wherein hybridized nucleic acid impurities comprise double stranded RNA.

60. The method of claim 58, wherein hybridized nucleic acid impurities comprise RNA:DNA hybrids.

61. The method of claim 58, wherein hybridized nucleic acid impurities comprise DNA or RNA that is an affinity ligand leached from oligonucleotide-based resins.

62. The method of claim 46, wherein the stationary phase is selected from a group consisting of: poly styrene divinylbenzene, polymethacrylate, crosslinked agarose, allyl dextran with N-N-bis acrylamide, silica, dextran, polyacrylamide, hydrophilic media, and hydrophobic media.

63. The method of claim 46, wherein the RNA transcript is the product of in vitro transcription using a non-amplified DNA template.

64. A method for characterizing an RNA transcript, comprising:
obtaining the RNA transcript of 300 to 10,000 nucleotides in length or is chemically modified; and
characterizing the RNA transcript using a procedure selected from the group consisting of chip-based capillary electrophoresis, agarose gel electrophoresis, analytical ultracentrifugation, and field flow fractionation.

65. The method of claim 64, wherein the RNA transcript is 700 to 3,000 nucleotides in length.

66. The method of claim 64, wherein the RNA transcript is 800 to 2,000 nucleotides or base pairs in length.

67. The method of claim 64, wherein the RNA transcript is a full length RNA transcript.

68. The method of claim 64, wherein the RNA transcript is chemically modified.

69. The method of claim 64, wherein the characterizing is performed by chipbased capillary electrophoresis.

70. The method of claim 69, wherein separation of the RNA transcript from impurities in a sample comprises separation based on hydrodynamic size and charge under denaturing conditions.

71. The method of claim 69, wherein chip-based capillary electrophoresis comprises steps of:
delivering the sample into a channel of a chip with an electrolyte medium;
applying an electric field to the chip that causes the RNA transcript and the impurities migrate through the channel, wherein the RNA transcript has a different electrophoretic mobility than the impurities such that the RNA transcript migrates through the channel at rate that is different from a rate at which the impurities migrate through the channel;
collecting from the chip the sample comprising the RNA transcript and one or more separate portions of the sample comprising the impurities; and
characterizing an aspect of at least one of the portion of the sample comprising the RNA transcript and the one or more separate portions of the sample comprising the impurities.

72. The method of claim 64, wherein characterizing at least one aspect comprises quantitative charge heterogeneity analysis.

73. The method of claim 72, wherein the electrophoretic mobility of the RNA transcript is proportional to an ionic charge the RNA transcript and inversely proportional to frictional forces in the electrolyte medium.

74. The method of claim 64, wherein the characterizing is performed by agarose gel electrophoresis.

75. The method of claim 74, wherein mRNA topology and hydrodynamic size are measured.

76. The method of claim 64, wherein the characterizing is performed by analytical ultracentrifugation.

77. The method of claim 76, wherein molecular weight distribution is measured, and wherein both equilibrium analytical ultracentrifugation and sedimentation analytical ultracentrifugation are used.

78. The method of claim 64, wherein the characterizing is performed by field flow fractionation.

79. The method of claim 64, wherein the RNA transcript is the product of in vitro transcription using a non-amplified DNA template.
